(19) United States
(12) Patent Application Publication
(10) Pub. No.: US 2026/0165767 A1
(43) Pub. Date: Jun. 18, 2026

(54) Duodenal and Jejunal Ablation with Improved Depth and Consistency of Ablation

(71) Applicant: Aqua Medical, Inc., Santa Ana, CA (US)

(72) Inventors: Scott McGill, San Ramon, CA (US); Virender K. Sharma, Paradise Valley, AZ (US)

(21) Appl. No.: 19/365,034

(22) Filed: Oct. 21, 2025

Related U.S. Application Data
(63) Continuation-in-part of application No. 18/593,883, filed on Mar. 2, 2024.
(60) Provisional application No. 63/488,106, filed on Mar. 2, 2023.
(60) Provisional application No. 63/596,196, filed on Nov. 3, 2023.
(60) Provisional application No. 63/618,313, filed on Jan. 6, 2024.
(60) Provisional application No. 63/710,552, filed on Oct. 22, 2024.
(60) Provisional application No. 63/797,416, filed on Apr. 30, 2025.

(51) Int. Cl. A61B 18/08; A61B 18/00; A61M 25/00

(52) U.S. Cl. CPC A61B 18/082; A61B 2018/00577; A61B 2018/00714; A61B 2218/005; A61M 25/0023; A61M 25/0045; A61M 25/007

(57) ABSTRACT

Ablation catheters and systems include flexible catheter tips with at least one positioning element and ports for delivery of an ablative agent to a target tissue. The positioning element is used to define a treatment zone and position the catheter proximate the target tissue for ablation. Ablative fluid is delivered to the target tissue at subtherapeutic, therapeutic, or supratherapeutic doses over different time periods, with rest periods between each dose, to cause effective ablation of the target tissue.

### CROSS-REFERENCE

The present application relies on U.S. Patent Provisional Application No. 63/797,416, titled “Duodenal and Jejunal Ablation with Improved Depth and Consistency of Ablation” and filed on Apr. 30, 2025, and U.S. Patent Provisional Application No. 63/710,552, of the same title and filed on Oct. 22, 2024, for priority, both of which are herein incorporated by reference in their entirety.

The present application is also a continuation-in-part application of U.S. patent application Ser. No. 18/593,883, titled “Duodenal Ablation with Improved Depth and Consistency of Ablation” and filed on Mar. 2, 2024, which relies on, for priority, U.S. Patent Provisional Application No. 63/618,313, titled “Vapor-Based Ablation Treatment Methods with Improved Treatment Volume Vapor Management” and filed on Jan. 6, 2024, U.S. Patent Provisional Application No. 63/596,196, of the same title and filed on Nov. 3, 2023, and U.S. Patent Provisional Application No. 63/488,106, of the same title and filed on Mar. 2, 2023.

All of the above referenced applications are herein incorporated by reference in their entirety.

### FIELD

The present specification relates to systems and methods configured to generate and deliver vapor for ablation therapy. More particularly, the present specification relates to systems and methods comprising flexible catheter positioning elements and/or tips with needles or ports for delivering ablation therapy to specific organ systems.

### BACKGROUND

Ablation, as it pertains to the present specification, relates to the removal or destruction of a body tissue, via the introduction of a destructive agent, such as radiofrequency energy, laser energy, ultrasonic energy, cryoagents, steam, or other forms or methods of generating heat. Ablation is commonly used to eliminate diseased or unwanted tissues, such as, but not limited to cysts, polyps, tumors, hemorrhoids, precancerous lesions and tissue, and other similar lesions.

Over the past decades several endoscopic therapies have been developed to treat Barrett's esophagus (BE) with early neoplasia. The current treatment strategy consists of endoscopic resection of visible abnormalities, followed by ablation therapy for residual flat BE. The most widely adopted ablation technique is radiofrequency ablation (RFA) which has proven to be effective, safe and durable. Nevertheless, there are several disadvantages that are presented with the use of RFA. RFA catheters lack a simple “through-the-scope” design and have to be mounted on or passed through alongside the endoscope. This is not only time-consuming but may require removal and reintroduction of the endoscope. Moreover, RFA may be technically difficult in an esophagus with an altered anatomy or scarring, which resulted from a previous endoscopic resection. Lastly, RFA is associated with clinically relevant post-procedural pain.

To overcome these limitations, a novel vapor-based endoscopic ablation system was developed which may serve as an alternative. This radiofrequency vapor ablation (RFVA) system (Aqua Medical Inc., Santa Ana, California, USA) induces thermal ablation through high temperature water steam (100° C.) without making direct contact with the target tissue. Vapor ablation has already been demonstrated to be safe and effective for the treatment of other medical conditions, such as lung emphysema, dysfunctional uterine bleeding, and benign prostatic hyperplasia.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. U.S. Pat. Nos. 9,561,068, 9,561,067, and 9,561,066 are hereby incorporated herein by reference. One problem that the above-mentioned steam-based ablation systems typically present is the potential for overheating and/or burning of healthy tissue. Steam passing through a channel within a body cavity heats surfaces of the channel and may cause exterior surfaces of the medical tool, other than the operational portion of the tool itself, to become excessively hot. As a result, physicians may unintentionally burn healthy tissue when external portions of the device, other than the operational end of the tool, accidentally contacts healthy tissue.

The effective use of steam often requires controllably exposing a volume of tissue to steam. However, prior art approaches to steam ablation either fail to sufficiently enclose a volume being treated, thereby insufficiently exposing the tissue, or excessively enclose a volume being treated, thereby dangerously increasing pressure and/or temperature within the patient's organ. Pressure sensors located on the catheter may help regulate energy delivery, but they are not necessarily reliable and represent a critical point of potential failure in the system. Therefore, among the several disadvantages of the conventional approaches to performing vapor-based ablation, foremost is the difficulty of controlling energy deposition to achieve uniform ablation in the treatment zone. Conventional vapor ablation systems may selectively add cooling fluid to control temperature in the treatment area and/or use specialized application components, such as balloons or nozzles. However, these approaches add substantial complexity to the system and typically fail to provide the required uniformity.

A lack of uniformity in ablation can cause certain portions of a treatment area to be insufficiently ablated, for example, a small fraction of the depth of the mucosa layer of the patient's duodenum, while concurrently causing certain portions of the treatment area to be excessively ablated, such as a substantial fraction of the depth of the serosa layer of the patient's duodenum.

In the human anatomy, the duodenum is the first part of the small intestine through which food passes and is initially digested, while the jejunum (middle portion of the small intestine) works on digesting the food further. The duodenum is mostly retroperitoneal (only the first 2-3 cm being intraperitoneal) and relatively fixed in place, as compared to other parts of the small intestine, and is therefore easier to ablate. The jejunum is entirely intraperitoneal and freely mobile, which makes it more difficult to control an ablation catheter tip and more difficult to position the catheter within. As a result, the jejunum has historically been more challenging to ablate, often leading to complications. Additionally, the duodenum is relatively shorter, measuring approximately 20 to 25 centimeters (cm), while the jejunum is much longer and can extend for up to 2.5 meters (m). Further, the duodenum has a characteristic C-shape, while the jejunum does not have a specific shape, but forms coils. The duodenum receives blood from both the celiac artery and superior mesenteric artery, while the jejunum is supplied solely by branches of the superior mesenteric artery.

Moreover there are histological differences between the duodenum and the jejunum. Brunner's glands are present in the submucosa of the duodenum, while they are absent in the jejunum. Circular folds (plicae circulares) are less prominent in the duodenum and more prominent as well as numerous in the jejunum. Villi are present in both but is generally longer and more numerous in the jejunum. The lymphoid tissue is less abundant in the duodenum and more abundant in the jejunum with occasional Peyer's patches. The jejunum has a thicker wall compared to the duodenum. The jejunum also has a wider lumen than the duodenum. These differences reflect the specialized functions of each segment of the small intestine, with the duodenum focused on mixing chyme with digestive secretions and the jejunum optimized for nutrient absorption.

Even the duodenojejunal flexure plays several important roles in digestion. The duodenojejunal flexure is an anatomical transition point from the upper to the lower gastrointestinal tract. It forms a sharp angle that helps control the passage of chyme from the duodenum into the jejunum. Its position at the end of the duodenum allows it to play a role in coordinating the mixing of chyme with pancreatic enzymes and bile before entering the jejunum for further digestion and absorption. Being retroperitoneal, the duodenojejunal flexure is less mobile than the jejunum, helping to stabilize the beginning of the jejunum. The suspensory muscle of the duodenum contracts to widen the angle of the flexure, facilitating the movement of intestinal contents from the duodenum into the jejunum. The sharp angle of the flexure may help prevent reflux of jejunal contents back into the duodenum. In abdominal surgeries, the duodenojejunal flexure serves as an important anatomical landmark, often identified by the location of the inferior mesenteric vein. Therefore, the duodenojejunal flexure's anatomical and functional properties contribute significantly to the overall digestive process by regulating the flow of intestinal contents and marking an important transition point in the small intestine.

The duodenum is involved in sensing nutrients, such as glucose, which triggers various physiological responses, and in particular plays a crucial role in glucose homeostasis. Research suggests that the duodenum may secrete proteins that induce insulin resistance. Some studies propose the existence of a “secretin” hormone in the duodenum that inhibits insulin secretion.

The jejunum also has a significant contribution in glucose regulation. The proximal jejunum (or the beginning part) is involved in nutrient sensing, which can influence hepatic glucose production. The jejunum is a primary site for glucose absorption, which affects postprandial glucose levels. In type 2 diabetes, glucose absorption and the expression/activity of glucose transporters SGLT1 and GLUT2 in enterocytes are increased, potentially contributing to hyperglycemia. The jejunum secretes incretin hormones like glucagon-like peptide-1 (GLP-1) and glucose-dependent insulinotropic polypeptide (GIP), which enhance insulin secretion. GLP-1 secretion from the jejunum is more sustained compared to the duodenum. The jejunum contributes to gastrointestinally induced glucose disposal (GIGD), a mechanism that clears glucose from circulation after oral ingestion. However, GIGD is reduced in type 2 diabetes patients.

Bariatric surgeries that bypass the duodenum and parts of the jejunum, such as Roux-en-Y gastric bypass (RYGB), biliopancreatic diversion (BPD), and partial jejunal diversion, have shown significant improvements in glucose regulation, glucose homeostasis and diabetes remission. RYGB primarily affects insulin secretion and hepatic glucose production, while BPD (which results in bypassing the duodenum) can lead to rapid normalization of insulin sensitivity. After RYGB, undigested food delivered to the jejunum may reduce hepatic glucose production. This suggests that the jejunum may secrete factors that affect insulin resistance and that it plays a role in glucose regulation. Duodenal-jejunal exclusion improves glucose tolerance through enhanced GLP-1 action, as demonstrated in animal studies. Duodenal-jejunal bypass surgery has been shown to restore sweet taste receptor-mediated glucose sensing and absorption, improving glycemic control. Duodenal-jejunal bypass surgery increases, but does not fully normalize, the β-cell response to glucose ingestion. Therefore, the duodenum and jejunum have distinct roles in glucose homeostasis, and their differential functions contribute to the complex pathophysiology of type 2 diabetes. Understanding these differences has led to innovative surgical approaches for diabetes treatment and continues to inform research into potential therapeutic targets. Additionally, glucose administration into the ileum (distal small intestine) compared to the duodenum results in enhanced GLP-1 secretion and a greater incretin effect in both healthy subjects and those with type 2 diabetes.

In addition, partial jejunal diversion (PJD) has emerged as a non-drug intervention to treat conditions such as obesity and Type 2 diabetes. PJD is a simple laparoscopic surgical procedure that involves creating a side-to-side anastomosis that allows a portion of nutrients to bypass the intact loop of bowel while the remaining portion of nutrients follows the common path of intestinal transit. The procedure partially diverts stomach contents in the small intestine, improving glycemic control with less weight loss than current bariatric metabolic surgeries. Bypassing part of the jejunum may alter jejunal nutrient sensing, which can influence hepatic glucose production. PJD leads to significantly lower fasting blood glucose levels and improved glucose tolerance as early as 2 weeks after surgery, with continued improvements over 12 months. PJD results in a significant increase in glucagon-like peptide-1 (GLP-1) secretion, as measured by the area under the curve (AUC) during oral glucose tolerance tests (OGTT). At 12 months post-PJD, patients show a significant increase in β-cell function, contributing to better glucose regulation. PJD leads to an increase in whole-body insulin sensitivity, improving glucose uptake and utilization. In animal studies, PJD has been shown to upregulate the expression of SGLT1 and GLUT1 in the alimentary limb, while downregulating GLUT2 expression. This may contribute to improved glucose handling. PET-CT studies in animals have shown increased [18F]-FDG uptake in the proximal jejunum after PJD, suggesting increased glucose accumulation in the alimentary limb. By diverting nutrient flow directly into the jejunum, PJD may alter jejunal nutrient sensing and glucose flux, contributing to improved glucose homeostasis. Unlike more invasive procedures like Roux-en-Y gastric bypass, PJD improves glucose tolerance without causing significant malabsorption or drastic changes in body weight. PJD leads to significant reductions in HbA1c levels, with 53.3% of patients achieving a >2% absolute reduction and 46.7% reaching HbA1c <7.0 % at 12 months post-surgery. Therefore, PJD offers numerous benefits in glycemic control.

There is limited direct information about how partial jejunal diversion (PJD) specifically impacts the gut microbiota, but some potential effects are inferred and presented herein based on related metabolic surgeries and their impacts on gut microbiota. For example, metabolic surgeries such as Roux-en-Y gastric bypass (RYGB) have been shown to cause shifts in gut microbiota composition. It is possible that PJD may also lead to changes in the relative abundance of different bacterial phyla and genera. After RYGB, there is often an increase in microbial richness and diversity. PJD might have a similar effect, potentially restoring microbial richness that is often lost in obesity and type 2 diabetes. RYGB has been associated with increases in certain bacterial groups, such as Faecalibacterium prausnitzii, which is linked to reduced low-grade inflammation in obesity and diabetes. PJD might also promote changes in specific beneficial bacteria. Changes in gut microbiota after metabolic surgeries are often associated with improvements in metabolic parameters. While not directly stated for PJD, the procedure does lead to improvements in glucose tolerance and insulin sensitivity, which could be partially mediated by changes in gut microbiota. PJD creates a side-to-side anastomosis that allows a portion of nutrients to bypass an intact loop of bowel. This altered nutrient flow could impact the microbial environment in different parts of the intestine, potentially leading to changes in microbial populations. Changes in bile acid metabolism, alterations in the intestinal microbiome, and improved beta cell function have been suggested as mechanisms contributing to improved glycemia after duodenal-jejunal bypass. Similar mechanisms might be at play in PJD, potentially involving the gut microbiota.

Bypass length in PJD is a factor that affects glucose tolerance. During a research, two different bypass lengths were tested in the rodent studies. In one case, an anti-mesenteric incision was made 30 cm beyond the ligament of Treitz and another 30 cm from the ileocecal junction. The other case was identical to the 30 cm version except the distal incision was made 18 cm from the ileocecal junction. Both 30 cm and 42 cm PJD versions resulted in significantly lower blood glucose excursions compared to sham surgery during a mixed-meal tolerance test (MMTT). The 30 cm PJD showed significantly lower blood glucose at 15 minutes post-MMTT. The 42 cm PJD showed significantly lower blood glucose at 120 minutes post-MMTT. The differences between 30 cm and 42 cm PJD were described as “subtle” overall. There was a trend for rats with 42 cm PJD to weigh less than those with 30 cm PJD. Fat absorption was reduced by approximately 10% in the 42 cm PJD group compared to sham, while fat absorption was unaffected in the 30 cm PJD group. In the human clinical study, the procedure involved creating a side-to-side anastomosis 100 cm from the ligament of Treitz and 250 cm from the ileocecal junction. This resulted in an estimated mean oral jejunum to oral ileum flow of 57%-43% respectively. Significant improvements in HbA1c, fasting blood glucose, and glucose tolerance were observed. These improvements were seen as early as 2 weeks post-surgery and continued over 12 months. Therefore, while both bypass lengths improved glucose tolerance in rodents, the longer bypass (42 cm) showed a trend towards greater weight loss and slightly reduced fat absorption. The human study used a single bypass length and showed significant improvements in glucose homeostasis, but did not directly compare different bypass lengths in humans.

Methods such as PJD or ‘bypass’ are traditionally advised to be performed at least 30 centimeters (cm) from the ligament of Treitz, rather than at the ligament itself. The reason is based on several anatomical and physiological considerations. These considerations include mobility of the intestine, preservation of duodenal function, surgical feasibility, optimization of metabolic effects, avoidance of complications, positive experimental evidence, and standardization. The optimal bypass length may vary depending on the specific goals of the procedure and individual patient factors. The 30 cm point appears to be a compromise that balances effectiveness with safety and feasibility in many cases. PJD offers several advantages. PJD avoids complications such as severe diarrhea and eliminates the blind limb seen with jejunoileal bypass (JIB). Additionally, PJD is potentially reversible and does not impose significant lifestyle alterations.

Most ablation methods and devices are configured to ablate the duodenum. The jejunum's functions in glucose absorption, hormone secretion, and nutrient sensing significantly impact glucose homeostasis in type 2 diabetes patients. Alterations in these functions contribute to the pathophysiology of the disease, while bypassing or modifying the jejunum's role can lead to improvements in glucose regulation. Therefore, it is desirable to be able to treat the jejunum. The jejunum is anatomically hard to reach and most ablation devices that are not placed at a target site through an endoscopic channel, are unable to reach the jejunum. Conventional ablation devices that are attempted to be pushed towards the jejunum pose a risk of catheter related injury to the jejunal wall.

Additionally, current endoscopic and surgical interventions for the treatment of Type 2 Diabetes Mellitus (T2DM), such as duodenal mucosal resurfacing (DMR) and bariatric surgeries (including RYGB and Duodenal-Jejunal Bypass), primarily target the post-ampullary duodenum (D2-D4). The rationale for this focus is based on its role in incretin hormone secretion (GLP-1, GIP) and glucose absorption. However, previous interventions have overlooked the potential metabolic regulatory role of the duodenal bulb (D1) due to complications associated with duodenal bulb interventions, such as hepatic abscesses and gastrointestinal bleeding. It is likely that the duodenal bulb plays a pivotal role in nutrient sensing, gastric emptying, and neurohormonal signaling, which are dysregulated in T2DM.

It is desirable to have steam-based ablation devices that integrate safety mechanisms into the device itself preventing unwanted burning during use and that can mimic the benefits of PJD through ablation of the jejunum. It is further desirable to be able to provide a way to better control the amount of steam to which a target tissue is exposed. It is also desirable to be able to control a pressure level within an enclosed volume without relying on a pressure sensor in the catheter itself. It is further desirable to expose the target tissue to steam without increasing the pressure of the exposed tissue. Also, there is a need for a vapor-based ablation system that can achieve effective ablation uniformity within a defined treatment area. Finally, it is also desirable to provide steam-based ablation systems and methods used to treat various conditions including metabolic syndrome, pre-cancerous or cancerous tissue in the esophagus, duodenum, bile duct, stomach, colon, and pancreas. It is further desirable to overcome the limitations of ablation that currently only span from the ligament of Treitz to a distance of approximately 30 cm from the ligament of Treitz; it is also desirable to be able to ablate the duodenojejunal flexure. What is therefore also needed is the application of ablative methods within the duodenal bulb to improve glucose homeostasis.

### SUMMARY

The present specification discloses a catheter assembly for vapor-based ablation therapy, comprising: a tubular body having a proximal end, a distal end, and an internal lumen extending therebetween defined by a longitudinal axis, wherein at least a portion of the internal lumen has a cross-section defined by opposed arcuate lobes separated by a central region that is constricted relative to the opposed arcuate lobes; a plurality of electrodes extending parallel to the longitudinal axis of the internal lumen and passing through the opposed arcuate lobes and central region of the lumen, wherein the plurality of electrodes is electrically coupled to a controller and configured to receive radiofrequency energy from the controller to heat a fluid within the lumen to form an ablative vapor; and ports extending along the tubular body that place the internal lumen in communication with an exterior of the catheter assembly, wherein the ports are positioned distal to the plurality of electrodes.

Optionally, the opposed arcuate lobes in combination with the constricted central region are configured to concentrate said heating of the fluid adjacent the plurality of electrodes while maintaining flow balance between the lobes to substantially uniformly generate the ablative vapor for discharge through one or more of said ports.

Optionally, the cross-section of the internal lumen is batwing-shaped. Optionally, the batwing-shaped lumen is configured to generate counter-rotational vortices of the ablative vapor in the two opposed arcuate lobes to equalize temperature.

Optionally, the opposed arcuate lobes comprise two symmetric lobes joined by a medial web having a thickness less than half of an outer wall thickness of the tubular body.

Optionally, the plurality of electrodes comprises thin-film conductive traces deposited on a polymeric layer. Optionally, the conductive traces comprise platinum-iridium or gold. Optionally, the conductive traces comprise a first set of anode traces and a second set of cathode traces and each of the first set of anode traces is separated from another one of the first set of anode traces by one of the second set of cathode traces.

Optionally, the plurality of electrodes comprise individual conductive traces of alternating polarity pairs positioned through the lobes to form a distributed resistive-heating zone.

Optionally, the constricted central region comprises a micro-channel configured to equalize pressure between the opposed arcuate lobes.

Optionally, a surface of the internal lumen comprises a hydrophilic coating.

Optionally, the plurality of electrodes is electrically isolated from each other by one or more dielectric ribs. Optionally, the one or more dielectric ribs are molded into the opposed arcuate lobes and/or the central region.

The present specification also discloses a vapor-ablation catheter comprising: an elongated flexible catheter shaft having a distal treatment section; at least one member positioned through the catheter shaft; a distal hub coupled to the at least one member; a proximal collar coupled to the at least one member; a plurality of wire members coupled to the distal hub and to the proximal collar, wherein the plurality of wire members are adapted to expand outwardly from a substantially linear configuration to a substantially cylindrical mesh defining a treatment zone; and a plurality of ports located within the substantially cylindrical mesh and configured to discharge vaporized ablative fluid into a volume defined by the substantially cylindrical mesh, wherein, upon expansion, the substantially cylindrical mesh is configured to center the catheter shaft within a hollow organ and establish a circumferential contact profile enabling uniform circumferential ablation when the vaporized ablative fluid is released through the ports.

Optionally, the substantially cylindrical mesh comprises a first wire mesh end cap, a second wire mesh end cap, and a central cylindrical wire mesh coupled to the first wire mesh end cap and the second wire mesh end cap. Optionally, the first wire mesh end cap, the second wire mesh end cap, and the central cylindrical wire mesh are adapted to expand concurrently and adapted to compress concurrently back into the substantially linear configuration.

Optionally, each of the plurality of wire members comprises a superelastic alloy, nitinol or stainless steel.

Optionally, the substantially cylindrical mesh comprises four to eight wire struts crossing at interlaced joints to form diamond-shaped openings.

Optionally, each of the plurality of wire members comprises an insulated proximal section and an exposed distal section adapted to reduce unwanted heating. Optionally, the vapor-ablation catheter further comprises an outer sheath positioned around an external surface of the catheter shaft, wherein the outer sheath is configured to move relative to the catheter shaft. Optionally, when in the substantially linear configuration, the substantially cylindrical mesh is covered by the outer sheath and wherein, when in the expanded configuration, the substantially cylindrical mesh is not covered by the outer sheath. Optionally, the substantially cylindrical mesh is configured to automatically expand upon retracting the outer sheath proximally relative to the catheter shaft. Optionally, the substantially cylindrical mesh is configured to automatically compress upon extending the outer sheath distally relative to the catheter shaft.

Optionally, the substantially cylindrical mesh defines a length of the treatment zone wherein said length is between 5 mm and 30 mm.

Optionally, the vapor-ablation catheter further comprises a cooling lumen extending parallel to the catheter shaft and adapted to maintain an external wall of the vapor ablation catheter below 45° C.

Optionally, at least one of the plurality of wire members comprises a temperature sensor, a pressure sensor, and/or impedance sensor.

Optionally, the substantially cylindrical mesh comprises a first wire mesh end cap and a second wire mesh end cap, wherein each of the first wire mesh end cap and the second wire mesh end cap comprises inter-wire gaps permitting 1-80% of the vaporized ablative fluid to flow out of the treatment zone.

Optionally, the distal hub comprises a rounded atraumatic tip.

Optionally, at least one of the proximal collar, the distal hub, and the substantially cylindrical mesh comprises at least one radiopaque marker.

Optionally, the proximal collar is configured to be slidable along the at least one member to adjust a degree of expansion of the substantially cylindrical mesh.

Optionally, the distal hub is configured to be slidable along the at least one member to adjust a degree of expansion of the substantially cylindrical mesh.

Optionally, the vapor-ablation catheter further comprises at least one filter element having pores and adapted to generate backpressure.

The present specification also discloses a method for treating at least one of excess weight, obesity, eating disorders, metabolic syndrome, dyslipidemia, diabetes, polycystic ovarian disease, fatty liver disease, non-alcoholic fatty liver disease, metabolic dysfunction-associated steatohepatitis, or non-alcoholic steatohepatitis disease by ablating intestinal tissue using a vapor ablation system, wherein the vapor ablation system comprises a catheter having at least one positioning element of cylindrical basket-shaped mesh structure configured to expand outward from the catheter, wherein, upon expansion, the at least one positioning element defines a portion of a treatment zone, wherein ports are positioned on the catheter and are configured to direct ablative fluid from within the catheter out toward said treatment zone, and wherein the vapor ablation system further comprises a controller having at least one processor in electrical communication with the catheter, the method comprising: using an endoscope to thread the catheter through a working channel of the endoscope; positioning the catheter in a patient's intestine at a position that is 15 centimeters from Ampulla of Vater; treating within the intestine, the treatment comprising: causing the at least one positioning element to expand and define the portion of the treatment zone; activating the controller, wherein, upon activation, the controller delivers a first fluid to the catheter and causes the catheter to heat the first fluid to form a first ablative fluid such that the first ablative fluid leaves the catheter through the ports over a first period of time, wherein the first ablative fluid delivered over the first period of time constitutes a dose of circumferential ablation; causing the at least one positioning element to at least partially collapse; moving the catheter distally by a length of the at least one positioning element; and repeating the treating within the intestine to form a plurality of treatment zones, wherein the repeating is performed until a distance of at most 150 centimeters from Ampulla of Vater is circumferentially ablated.

Optionally, each dose is delivered at an energy level of 250 Joules.

Optionally, the first period of time is 3 seconds.

Optionally, each of the plurality of treatment zones at least partially overlaps with a neighboring treatment zone during each of the repeating.

Optionally, the dose is preset at the controller.

Optionally, the positioning further comprises positioning the catheter in the patient's intestine at a position that is at least 2 centimeters post ligament of Treitz of the patient and wherein the repeating is performed until a distance of at most 125 centimeters post ligament of Treitz.

Optionally, the method further comprises applying at least two doses and at most three doses of ablative fluid to each of the plurality of treatment zones, wherein each of the plurality of treatment zones overlaps with a neighboring one of the plurality of treatment zones such that they share in a range of 5% to 95% of their tissue in common.

Optionally, the method further comprises measuring the patient's fasting glucose before performing the method and approximately seven days after performing the method, wherein the patient's fasting glucose over approximately seven days after performing the method daily is at least 30% less than the patient's fasting glucose before performing the method.

Optionally, the method further comprises measuring the patient's post-prandial glucose before performing the method and approximately seven days after performing the method, wherein the patient's post-prandial glucose approximately seven days after performing the method is at least 19% less than the patient's post-prandial glucose before performing the method.

Optionally, each of the plurality of treatment zones is defined by a plurality of consecutively positioned annular rings, wherein each of the plurality of consecutively positioned annual rings has an internal surface area, and wherein, after the method, at least 60% of the internal surface area of each of the plurality of consecutively positioned annual rings is effectively ablated.

The present specification also discloses a method for treating at least one of excess weight, obesity, eating disorders, metabolic syndrome, dyslipidemia, diabetes, polycystic ovarian disease, fatty liver disease, non-alcoholic fatty liver disease, metabolic dysfunction-associated steatohepatitis, or non-alcoholic steatohepatitis disease by ablating intestinal tissue using a vapor ablation system, wherein the vapor ablation system comprises a catheter having at least one positioning element of cylindrical basket-shaped mesh structure configured to expand outward from the catheter, wherein, upon expansion, the at least one positioning element defines a portion of a treatment zone, wherein ports are positioned on the catheter and are configured to direct ablative fluid from within the catheter out toward said treatment zone, and wherein the vapor ablation system further comprises a controller having at least one processor in electrical communication with the catheter, the method comprising: using an endoscope to thread the catheter through a working channel of the endoscope; positioning the catheter in a patient's intestine at a position that is at most 150 centimeters from Ampulla of Vater; treating within the intestine, the treatment comprising: causing the at least one positioning element to expand and define the portion of the treatment zone; activating the controller, wherein, upon activation, the controller delivers a first fluid to the catheter and causes the catheter to heat the first fluid to form a first ablative fluid such that the first ablative fluid leaves the catheter through the ports over a first period of time, wherein the first ablative fluid delivered over the first period constitutes a first dose of circumferential ablation; causing the at least one positioning element to at least partially collapse; moving the catheter proximally by a length of the at least one positioning element; repeating the treating within the intestine to form a plurality of treatment zones, wherein the repeating is performed until a distance of at least 15 centimeters from Ampulla of Vater is circumferentially ablated; moving the catheter proximally to a distance of at least 2 centimeters on a proximal side from Ampulla of Vater; and activating the controller, wherein, upon activation, the controller delivers a second fluid to the catheter and causes the catheter to heat the second fluid to form a second ablative fluid such that the second ablative fluid leaves the catheter through the ports over the first period of time, wherein the second ablative fluid delivered over the first period constitutes a second dose of circumferential ablation.

Optionally, each of the first dose and the second dose is delivered at an energy level of 250 Joules.

Optionally, the first period of time is 3 seconds.

Optionally, each of the plurality of treatment zones at least partially overlaps with a neighboring treatment zone during each of the repeating.

Optionally, each of the first dose and the second dose is preset at the controller.

Optionally, the positioning further comprises positioning the catheter in the patient's intestine at a position that is at most 125 centimeters post ligament of Treitz of the patient and wherein the repeating is performed until a distance of at least 2 centimeters post ligament of Treitz.

Optionally, the method further comprises applying at least two first doses and at most three first doses of ablative fluid to each of the plurality of treatment zones, wherein each of the plurality of treatment zones overlaps with a neighboring one of the plurality of treatment zones such that they share in a range of 5% to 95% of their tissue in common.

Optionally, the method of further comprises measuring the patient's fasting glucose before performing the method and approximately seven days after performing the method, wherein the patient's fasting glucose over approximately seven days after performing the method daily is at least 30% less than the patient's fasting glucose before performing the method.

Optionally, the method further comprises measuring the patient's post-prandial glucose before performing the method and approximately seven days after performing the method, wherein the patient's post-prandial glucose approximately seven days after performing the method is at least 19% less than the patient's post-prandial glucose before performing the method.

Optionally, each of the plurality of treatment zones is defined by a plurality of consecutively positioned annular rings, wherein each of the plurality of consecutively positioned annual rings has an internal surface area, and wherein, after the method, at least 60% of the internal surface area of each of the plurality of consecutively positioned annual rings is effectively ablated.

The present specification also discloses a method for treating Type 2 Diabetes Mellitus (T2DM) or associated metabolic dysfunctions by ablating duodenal bulb tissue using a vapor ablation system, wherein the vapor ablation system comprises a catheter having at least one positioning element of cylindrical basket-shaped mesh structure configured to expand outward from the catheter, wherein, upon expansion, the at least one positioning element defines a portion of a treatment zone, wherein ports are positioned near a distal end of the catheter and are configured to direct ablative fluid from within the catheter out toward said treatment zone, and wherein the vapor ablation system further comprises a controller having at least one processor in electrical communication with the catheter, the method comprising: inserting the catheter through a working channel of an endoscope; positioning the distal end of the catheter within the duodenal bulb; and ablating the duodenal bulb, the ablation comprising: expanding the positioning element to define the treatment zone; activating the controller to deliver a dose of energy to generate vaporized ablative fluid, wherein the vapor is directed into the treatment zone to achieve circumferential ablation of the duodenal bulb tissue; repeating the ablation to deliver at least one additional dose of vapor to the duodenal bulb, wherein consecutive doses are separated by a pause of at least 5 seconds; and collapsing the positioning element and withdrawing the catheter upon completing the ablation.

Optionally, each dose of energy is between 200 Joules and 500 Joules.

Optionally, each dose of energy is delivered over a period of 3 seconds.

Optionally, the ablative fluid comprises saline and the vapor is generated by heating the saline within the catheter using radiofrequency energy.

Optionally, the treatment zone defined by the positioning element is approximately 25 millimeters (mm) in length.

Optionally, the vapor ablation system is configured to selectively ablate mucosal tissue of the duodenal bulb while preserving the submucosal and adjacent structures.

Optionally, the method further comprises applying suction to bring the duodenal bulb tissue into contact with the positioning element before vapor delivery.

The present specification also discloses a method for treating Type 2 Diabetes Mellitus (T2DM) or associated metabolic dysfunctions by ablating intestinal tissue using a vapor ablation system, wherein the vapor ablation system comprises a catheter having at least one positioning element of cylindrical basket-shaped mesh structure configured to expand outward from the catheter, wherein, upon expansion, the at least one positioning element defines a portion of a treatment zone, wherein ports are positioned on the catheter and are configured to direct ablative fluid from within the catheter out toward said treatment zone, and wherein the vapor ablation system further comprises a controller having at least one processor in electrical communication with the catheter, the method comprising: using an endoscope to thread the catheter through a working channel of the endoscope; positioning the catheter in a patient's intestine at a position that is 15 centimeters from Ampulla of Vater; and treating within the intestine, the treatment comprising: causing the at least one positioning element to expand and define the portion of the treatment zone; activating the controller for suctioning to bring the duodenal and/or jejunal tissue into direct contact with the positioning element; activating the controller to deliver a first fluid to the catheter and cause the catheter to heat the first fluid to form a first ablative fluid such that the first ablative fluid leaves the catheter through the ports over a first period of time, wherein the first ablative fluid delivered over the first period of time constitutes a dose of circumferential ablation; causing the at least one positioning element to at least partially collapse; moving the catheter distally by a length of the at least one positioning element; and repeating the treating within the intestine to form a plurality of treatment zones, wherein the repeating is performed until a distance of at most 70 centimeters from Ampulla of Vater within Duodenum, ligament of Treitz, and at most 55 centimeters of Jejunum, is circumferentially ablated.

Optionally, each dose is delivered at an energy level of 250 Joules.

Optionally, the first period of time is 3 seconds.

Optionally, each of the plurality of treatment zones at least partially overlaps with a neighboring treatment zone during each of the repeating.

Optionally, the dose is preset at the controller.

Optionally, the method further comprises applying at least two doses and at most three doses of ablative fluid to each of the plurality of treatment zones, wherein each of the plurality of treatment zones overlaps with a neighboring one of the plurality of treatment zones such that they share in a range of 5% to 95% of their tissue in common.

Optionally, each of the plurality of treatment zones is defined by a plurality of consecutively positioned annular rings, wherein each of the plurality of consecutively positioned annual rings has an internal surface area, and wherein, after the method, at least 60% of the internal surface area of each of the plurality of consecutively positioned annual rings is effectively ablated.

The present specification also discloses a method for treating Type 2 Diabetes Mellitus (T2DM) or associated metabolic dysfunctions by ablating intestinal tissue using a vapor ablation system, wherein the vapor ablation system comprises a catheter having at least one positioning element of cylindrical basket-shaped mesh structure configured to expand outward from the catheter, wherein, upon expansion, the at least one positioning element defines a portion of a treatment zone, wherein ports are positioned on the catheter and are configured to direct ablative fluid from within the catheter out toward said treatment zone, and wherein the vapor ablation system further comprises a controller having at least one processor in electrical communication with the catheter, the method comprising: using an endoscope to thread the catheter through a working channel of the endoscope; positioning the catheter in a patient's intestine at a position that is within a jejunum; and treating within the intestine, the treatment comprising: causing the at least one positioning element to expand and define the portion of the treatment zone; activating the controller for suctioning to bring the duodenal and/or jejunal tissue into direct contact with the positioning element; activating the controller to deliver a first fluid to the catheter and cause the catheter to heat the first fluid to form a first ablative fluid such that the first ablative fluid leaves the catheter through the ports over a first period of time, wherein the first ablative fluid delivered over the first period of time constitutes a dose of circumferential ablation; causing the at least one positioning element to at least partially collapse; moving the catheter proximally by a length of the at least one positioning element; and repeating the treating within the intestine to form a plurality of treatment zones, wherein the repeating is performed until a distance of at most 70 centimeters from Ampulla of Vater within Duodenum, ligament of Treitz, and at most 55 centimeters of Jejunum, is circumferentially ablated.

Optionally, each of the first dose and the second dose is delivered at an energy level of 250 Joules.

Optionally, the first period of time is 3 seconds.

Optionally, each of the plurality of treatment zones at least partially overlaps with a neighboring treatment zone during each of the repeating.

Optionally, each of the first dose and the second dose is preset at the controller.

Optionally, the positioning further comprises positioning the catheter in the patient's intestine at a position that is at most 125 centimeters post ligament of Treitz of the patient and the repeating is performed until a distance of at least 2 centimeters post ligament of Treitz.

Optionally, the method further comprises applying at least two first doses and at most three first doses of ablative fluid to each of the plurality of treatment zones, wherein each of the plurality of treatment zones overlaps with a neighboring one of the plurality of treatment zones such that they share in a range of 5% to 95% of their tissue in common.

Optionally, each of the plurality of treatment zones is defined by a plurality of consecutively positioned annular rings, wherein each of the plurality of consecutively positioned annual rings has an internal surface area, and wherein, after the method, at least 60% of the internal surface area of each of the plurality of consecutively positioned annual rings is effectively ablated.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings, wherein:

FIG. 1A illustrates an ablation system, in accordance with embodiments of the present specification;

FIG. 1B illustrates the treatment end of a catheter comprising an outer sheath that, when pulled proximally toward the clinician, unveils positioning elements and a wire mesh structure connecting the two positioning elements;

FIG. 1C illustrates an exemplary electrode configuration, in accordance with some embodiments of the present specification;

FIG. 1D illustrates an electrode configuration circuit, in accordance with some embodiments of the present specification;

FIG. 1E illustrates tubing used to encase the electrode configurations of the embodiments of the present specification;

FIG. 1F illustrates different views of a tubing with a batwing-shaped internal lumen comprising an electrode configuration, in accordance with some embodiments of the present specification;

FIG. 2A illustrates the treatment end of a catheter comprising a positioning element with a wire mesh structure forming a basket-like shape, in accordance with some embodiments of the present specification;

FIG. 2B illustrates a direction of movement of an outer sheath of the catheter over first wire or member, in accordance with some embodiments of the present specification;

FIG. 2C shows an outer sheath positioned over and encompassing the collapsed configuration of the positioning element;

FIG. 2D illustrates a front cross-section view of a positioning element of a catheter, in accordance with some embodiments of the present specification;

FIG. 3 illustrates an exemplary embodiment of a vapor ablation system having a catheter with a basket shaped positioning element, in accordance with some embodiments of the present specification;

FIG. 4 illustrates positioning of a catheter having a basket shaped positioning element within a jejunum, in accordance with some embodiments of the present specification;

FIG. 5 is a flow chart illustrating an exemplary method of using vapor ablation devices of FIGS. 2A, 2B, 2C, 3, and 4 for jejunal ablation, in accordance with some embodiments of the present specification;

FIG. 6 is a flow chart illustrating another exemplary method of using vapor ablation devices of FIGS. 2A, 2B, 2C, 3, and 4 for jejunal ablation, in accordance with some embodiments of the present specification;

FIG. 7 is a graph illustrating daily fingerstick data of glucose measurement showing the readings for morning and evening, before the treatment and after the treatment, in accordance with the embodiments of the present specification;

FIG. 8 is a flow chart detailing an exemplary method of using the vapor ablation devices described with respect to FIGS. 2A, 2B, 2C, 3, and 4 for duodenal and jejunal ablation, in accordance with some embodiments of the present specification;

FIG. 9 is a digestive system showing the positioning of a clip to mark the proximal end or start of the treatment area, in accordance with some embodiments of the present specification;

FIG. 10 is a flow chart detailing an exemplary method of using the vapor ablation devices described with respect to FIGS. 2A, 2B, 2C, 3, and 4 for duodenal and jejunal ablation, in accordance with some embodiments of the present specification;

FIG. 11 is a flow chart detailing an exemplary method of using the vapor ablation devices described with respect to FIGS. 2A, 2B, 2C, 4, and 4 for duodenal bulb ablation, in accordance with some embodiments of the present specification;

FIG. 12 illustrates an anatomical view of a duodenal bulb, comprising a segment between an ampulla of Vater and a pylorus; and

FIG. 13 is a comparative graphical representation of HbA1C levels measured at a baseline, after 1 month, and after 3 months post-intervention using various combinations of intestinal ablation techniques in accordance with embodiments of the present specification.

### DETAILED DESCRIPTION

Embodiments of the present specification provide ablation systems and methods for treating various indications including, but not limited to, pre-cancerous or cancerous tissue in the esophagus, duodenum, bile duct, and pancreas. In various embodiments, steam, generated by heating saline, is used as an ablative agent. In various embodiments, the ablation systems include a generator for generating an ablative agent (steam generator), comprising a source for providing a fluid (saline) for conversion to a vapor (steam) and a catheter for converting and delivering said steam, wherein the catheter comprises at least one electrode embedded in a central lumen of the catheter and configured to function as a heating chamber to convert the saline to steam. The ablation systems further include an attachment at a distal end of the catheter, wherein the attachment comprises at least one of a needle, cap, hood, or disc. The attachment is configured to direct the delivery of ablative agent. The catheters may further include positioning elements to position the catheter for optimal steam delivery. The attachments and positioning elements are configured to create seals and form enclosed treatment volumes for the delivery of steam and ablation of target tissues. In embodiments, the ablation systems and methods of the present specification are configured to enclose an area or volume of tissue with at least one positioning attachment, fill that area or volume with vapor, allow the temperature in the area or volume to rise above 100° C., and then let the additional vapor escape, maintaining the temperature above 100° C. for a predetermined duration of time and the pressure in the area or volume less than 5 atm to allow the vapor to condense and ablate the tissue. The various embodiments described herein provide effective ablation methods and systems, which cause necrosis of tissue cells.

Embodiments of the present specification additionally provide methods and systems for the ablation of the duodenal bulb (D1) for treatment of Type 2 Diabetes Mellitus (T2DM). D1 differs significantly from the post-ampullary duodenum (D2-D4) in terms of anatomical structure, blood supply, and function, therefore making it a unique target for metabolic intervention. The blood in D1 is primarily supplied by the gastroduodenal artery, whereas D2-D4 receive a dual blood supply from the celiac trunk and superior mesenteric artery. Further, D1 has fewer Brunner's glands compared to D2, making it more susceptible to acid exposure and ulcer formation. Unlike D2-D4, the duodenal bulb plays a pivotal role in initial nutrient sensing, regulating gastric emptying, and neurohormonal signaling, which are all dysregulated in T2DM patients.

Radiofrequency (RF) vapor ablation methods and systems are used to modulate metabolic pathways and improve glycemic control in patients with T2DM. Ablative therapy in this region using the embodiments of the present specification significantly improves glucose homeostasis. The ablation of duodenal bulb modulates early nutrient sensing by disrupting aberrant duodenal nutrient sensing that contributes to insulin resistance. Further, ablation of the duodenal bulb modulates gastric emptying to prevent rapid glucose absorption and excessive postprandial hyperglycemia. Also, the ablation enhances neurohormonal feedback to suppress hepatic glucose output.

Configurations for the various catheters of the ablation systems of the embodiments of the present specification may be different based on the tissue or organ systems being treated. For example, in some embodiments, catheters for esophageal and duodenal ablation are similar, with the exception that the spacing between two positioning elements, positioned at distal and proximal ends of a distal portion of the catheter with at least one vapor delivery port between the two positioning elements, may be greater for esophageal applications (approximately 1-20 cm) than for duodenal applications (approximately 1-10 cm). Distribution and depth of ablation provided by the systems and methods of the present specification are dependent on the duration of exposure to steam, the ablation size, the temperature of the steam, the contact time with the steam, and the tissue type. In some embodiments, an outer wall of the catheter contains a cooling element within a lumen, such as a cooling liquid, to limit the maximum temperature (cool) of the outer surfaces of the catheter.

In some embodiments, a patient is treated in a two-step process to ensure complete or near complete ablation of a target tissue. In some embodiments, a patient is first treated with a catheter having two positioning elements—a distal positioning element that is initially deployed followed by a proximal positioning element deployed thereafter, and a tube length with at least one port positioned between the two positioning elements, thereby enabling wide area circumferential ablation. The positioning elements may be a balloon, a disc, or any other structure. A first seal is optionally created by contact of the periphery of the positioning elements with a patient's tissue at said distal and proximal positioning elements. The first seal may completely or partially seal and results in the formation of an enclosed first treatment volume, bounded by the distal positioning element at the distal end, the proximal positioning element at the proximal end, and the walls of the patient's tissue, such as the esophagus or duodenum, on the sides. Ablative energy, in the form of steam, is then delivered by the catheter via the ports into the first treatment volume, where it condenses and contacts the patient's tissue for circumferential ablation and cannot escape from the distal or proximal ends as it is blocked by the positioning elements or, alternatively, controllably escapes from the distal or proximal ends based on the configuration of the positioning elements, as further described below.

After ablation is performed using the catheter with two positioning elements, the ablation area is examined by the physician. Upon observing the patient, the physician may identify patches of tissue requiring focused ablation. A second step is then performed, wherein a second catheter with a needle or cap, hood, or disc attachment on the distal end is passed through an endoscope and used for focal ablation. The needle provides for directed, focal ablation and the cap, hood, or disc attachment encloses the focal ablation area, creating a second seal and an enclosed second treatment volume for ablation of the tissue. The seal is created by positioning at least a portion of a periphery of the cap, hood, or disc attachment in contact with a surface of a patient's tissue, such as the esophagus or duodenum, such that a portion of the patient's tissue is positioned within an area circumscribed by the attachment. In embodiments, the seal is a complete seal or a partial seal. A second treatment volume, configured to receive steam and bounded by the sides of the attachment and said circumscribed portion of patient tissue, is created when the seal is formed. Ablative energy, in the form of steam, is then delivered via the catheter by at least one port at the distal tip of the catheter into the second treatment volume, where it condenses and contacts the patient's tissue for focal ablation and cannot escape as it is bounded by the attachment or, alternatively, controllably escapes from the attachment based on the configuration of the attachment, as further described below. In one embodiment, the flow rate of vapor out of the enclosed, or partially enclosed, volume is a predefined percentage of the flow rate of vapor into the enclosed, or partially enclosed, volume from the catheter ports, where the predefined percentage is in a range of 1% to 80%, preferably less than 50%, and more preferably less than 30%. The at least one port is positioned at a distal end of the catheter such that it exits into the second treatment volume when the attachment is positioned.

During both the first and second steps, when creating the enclosed first and second treatment volumes, it is preferred to avoid creating a perfect (100%) seal. A perfect seal would trap air in the treatment volume. The trapped air would not be hot, relative to the steam used for ablation, and, therefore, would create ‘cold air pockets’ which act as a heat sink, sapping a portion of the thermal ablation energy of the steam and resulting in uneven distribution of the ablative energy of the steam. Creating less than a perfect seal allows for the air to be pushed out of the treatment volume, through a gap in the seal, as steam is delivered into the treatment volume.

Additionally, as the temperature in the treatment volume increases, no steam escapes until the temperature is greater than or equal to 100° C., at which point steam condensation stops and the steam is allowed to escape through the gap, preventing excessive pressurization of the treatment volume. In some embodiments, the generation of steam by heating saline is stopped by switching off the power to electrodes that generate heat until a time when a temperature of the ablation zone decreases to less than 45° C. or decreases by more than 25% from the peak temperature (such as for example, greater than or equal to 100° C.) during the ablation. In some embodiments, the catheter includes a filter with micro-pores which provides back pressure to the delivered steam, thereby pressurizing the steam as it enters the treatment volume from the catheter. The predetermined size of micro-pores in the filter determine the backpressure and hence the temperature of the steam being generated. During ablation with the attachment with two positioning elements, in various embodiments, a gap, or less than perfect seal, is positioned only at the distal positioning element, only at the proximal positioning element, or at both the distal and proximal positioning elements.

To create the gaps or less than perfect seals and allow air to leak or be pushed out of the treatment volumes, embodiments of the present specification provide positioning elements or attachments that have a range of 40% to 99% of their surface area in contact with the patient tissue. In embodiments, a surface area of a cross-sectional slice along a plane where a positioning element or attachment contacts the tissue is in a range of 20% to 99%. A low value, such as of 20%, represents an extremely porous seal, indicates that spacing exists between the positioning element or attachment and the tissue or that the positioning element or attachment includes voids therein, while a high value, such as 99%, represents a near perfect seal. Additionally, the first and second seals are considered low pressure seals, wherein pressure within the first and second treatment volumes formed by the seals is less than 5 atm and usually close to 1 atm. Therefore, as the pressure rises above a predetermined pressure level, the seal breaks and the heated air or vapor is allowed to escape, thereby obviating the need for a pressure sensor in the catheter itself.

In embodiments, one or more of the positioning elements or attachments are configured such that they permit a range of flow out of the treatment volumes enclosed by the two positioning elements or attachment. The permissible flow out is a function of steam flow into the enclosed volume, thereby acting as a relief valve and allowing for the maintenance of a desired pressure range (less than 5 atm) without regulation from the steam generator itself. In some embodiments, the positioning element or attachment comprises a plurality of spaces within the surface area of the positioning element or attachment and/or between the periphery of the positioning element or attachment and the tissue sufficient to permit a flow of fluid out of the enclosed volume in a range of 1 to 80% of the steam input flowrate to maintain the pressure level within the enclosed volume at less than 5 atm without regulation from the steam generator.

In some embodiments, the enclosed volume ranges from 3 cubic centimeters (cc) to 450 cc, when a surface area of mucosa to be ablated ranges from 5 square centimeter (cm²) to 200 cm².

In embodiments, one or more of the positioning elements or attachment are deformable over the course of treatment. Positioning elements and attachments in accordance with the embodiments of the present specification are designed to physically modify or deform when a pressure in the treatment volume increases above 10% of a baseline pressure, therefore effectively acting as a pressure relief valve. As a result of the ability to deform, the flow out of the volume enclosed by the two positioning elements or attachment is variable. In an exemplary embodiment, only a small portion, if any, of flow out of the enclosed volume is blocked at the beginning of therapy. The percentage of flow that is blocked decreases over the course of the therapy, thereby increasing leakiness, due to pressure changes. In some embodiments, assuming a positioning element or attachment blocks flow out of an enclosed volume (or has the cross-sectional area covered) in a range of 100% (total flow blockage or total cross section covered) to 20% (only 20% of flow blocked or only 20% of cross sectional area covered) at the start of treatment, the percentage changes during treatment where the amount of blockage/cross sectional area is decreased by 1% to 25% relative to the starting percentage. In various embodiments, as previously stated, it is preferred that pressure sensors are not included in the catheter itself to reduce costs and possible sensor failure. Therefore, the deformable positioning elements naturally act as relief valves, without requiring active pressure sensing.

In embodiments relating to methods and systems for the ablation of the duodenal bulb, a catheter-based RF vapor ablation device is used for targeted thermal modulation of duodenal mucosa. A patient is sedated before the endoscopic catheter is introduced into the duodenum through an endoscope channel. The RF vapor ablation device is positioned and deployed within the duodenal bulb. Controlled energy delivery is applied to ablate the duodenal mucosa while maintaining safety thresholds. The dose of energy delivered ranges from 200 J-500 J per application. Multiple applications can be applied after pausing for at least 5 seconds and for up to 1 minute, to achieve the desired therapeutic effect. In embodiments, the devices employ smart RF generators that are configured to precisely study the dosimetry to ensure the controlled delivery of energy and prevent excessive tissue damage. Endoscopic monitoring and imaging are conducted in real time for accurate positioning and targeting of ablation. Additionally, post-procedure monitoring is performed to ensure mucosal healing and improved metabolic response as measured by HbA1C, CGM, blood sugar levels, and HOMA-IR values.

In various embodiments, the ablation devices and catheters described in the present specification are used in conjunction with any one or more of the heating systems described in U.S. patent application Ser. No. 14/594,444, entitled “Method and Apparatus for Tissue Ablation”, filed on Jan. 12, 2015 and issued as U.S. Pat. No. 9,561,068 on Feb. 7, 2017, which is herein incorporated by reference in its entirety.

“Treat,” “treatment,” and variations thereof refer to any reduction in the extent, frequency, or severity of one or more symptoms or signs associated with a condition.

“Duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject.

“Period” refers to the time over which a “dose” of stimulation is administered to a subject as part of the prescribed treatment plan.

The term “effective ablation” is defined as the application of energy to tissue at a sufficient energy level so as to cause necrosis of tissue cells. A “sufficient energy level” may be achieved by modulating the temperature or thermal heat content of the vapor, by modulating the amount of time the tissue is subjected to vapor, and/or by appropriately configuring the vapor distribution and control components, such as the location of the fluid heating component within the catheter lumen, the location and relative distribution of ports along the catheter and the positioning elements.

The term “and/or” means one or all of the listed elements or a combination of any two or more of the listed elements.

In the description and claims of the application, each of the words “comprise” “include” and “have”, and forms thereof, are not necessarily limited to members in a list with which the words may be associated. The terms “comprises” and variations thereof do not have a limiting meaning where these terms appear in the description and claims.

Unless otherwise specified, “a,” “an,” “the,” “one or more,” and “at least one” are used interchangeably and mean one or more than one.

As described below, the sensors and components described in the present specification include key functionalities due to the unique combination of dimensions, thicknesses, flexibility, materials used, and positioning of certain elements. The key functionalities include, but are not limited to the key benefits as described herein. Therefore, the use and combination of these parameters should not be construed as mere design choices and, rather, should be accorded patentable weight. It should also be noted that the various components described herein may be used with any other component as described herein, in any combination or order, even if not described with respect to certain embodiments. Further, it should be noted that the various parameters described herein may apply without restriction to the embodiments in which they are described. Therefore, the components and parameters described throughout this specification are interchangeable and may be combined to achieve the objectives of the present invention and not limited to the specific embodiments.

The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements.

The term “vapor generation system” refers to any or all of the heater or induction-based approaches to generating steam from water described in this application.

For any method disclosed herein that includes discrete steps, the steps may be conducted in any feasible order. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range necessarily resulting from the standard deviation found in their respective testing measurements.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, liquefaction and absorption of the ablated tissue. The dose and manner of treatment can be adjusted based on the type of tissue and the depth of ablation needed. The ablation device can be used not only for the treatment of cardiac arrhythmias, Barrett's esophagus and esophageal dysplasia, flat colon polyps, gastrointestinal bleeding lesions, endometrial ablation, pulmonary ablation, but also for the treatment of any mucosal, submucosal or circumferential lesion, such as inflammatory lesions, tumors, polyps and vascular lesions. The ablation device can also be used for the treatment of focal or circumferential mucosal or submucosal lesions of any hollow organ or hollow body passage in the body. The hollow organ can be one of gastrointestinal tract, pancreaticobiliary tract, genitourinary tract, respiratory tract or a vascular structure such as blood vessels. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferro- or ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

In the embodiments of the present specification, ablative fluid preferably means heated vapor but can include cryogenic fluid as well.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

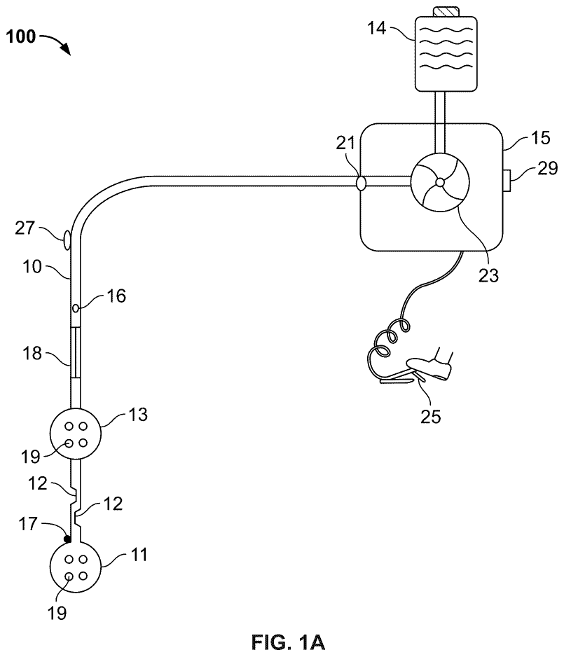

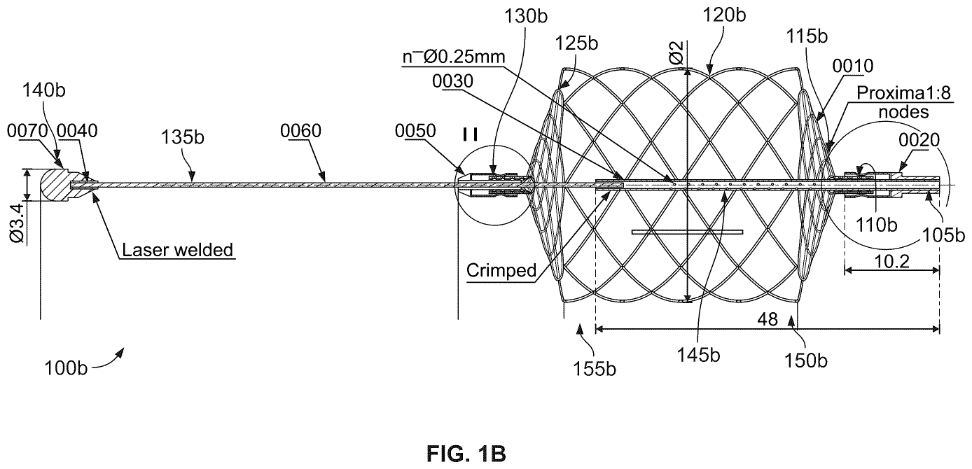

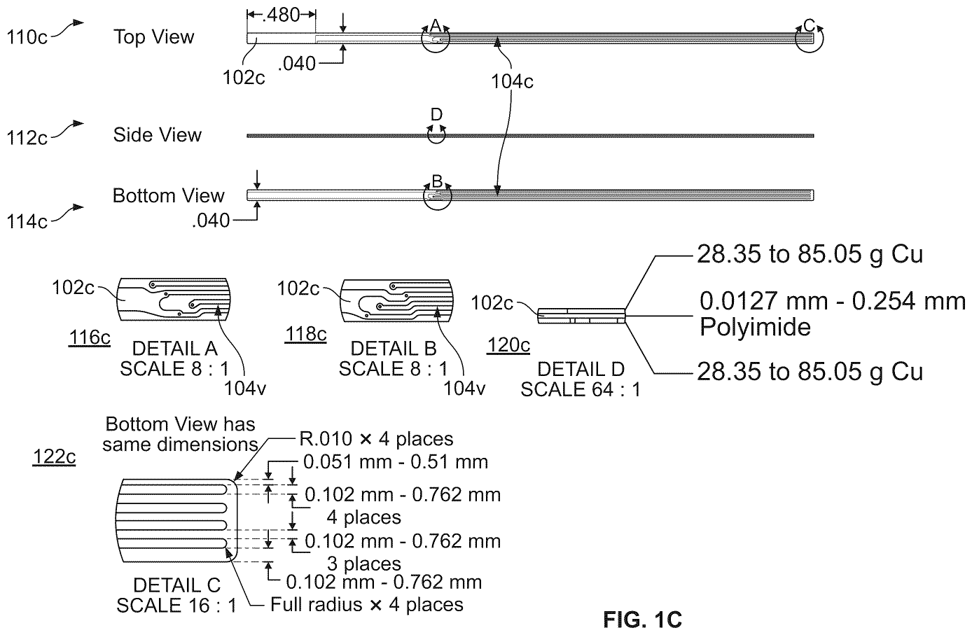

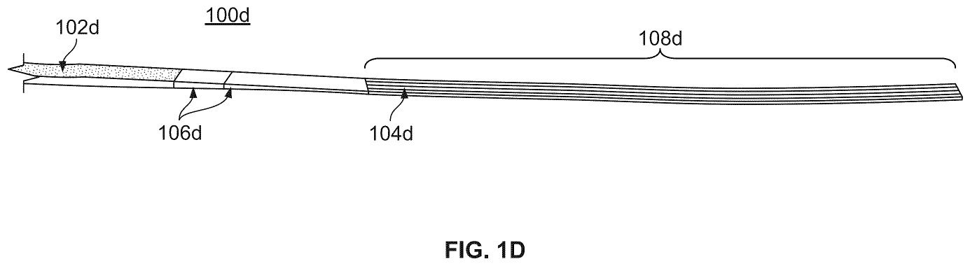

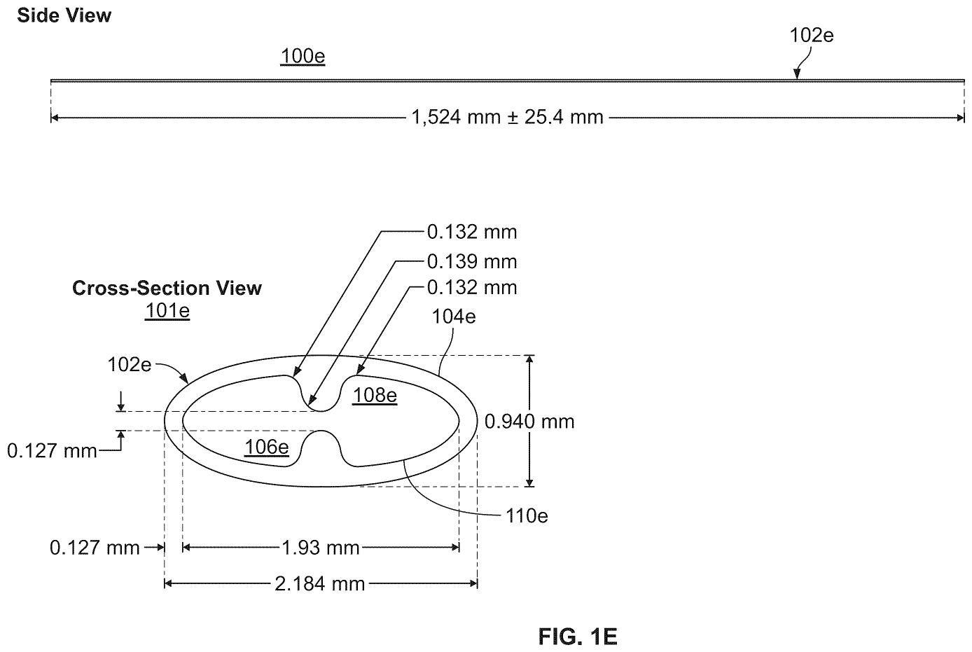

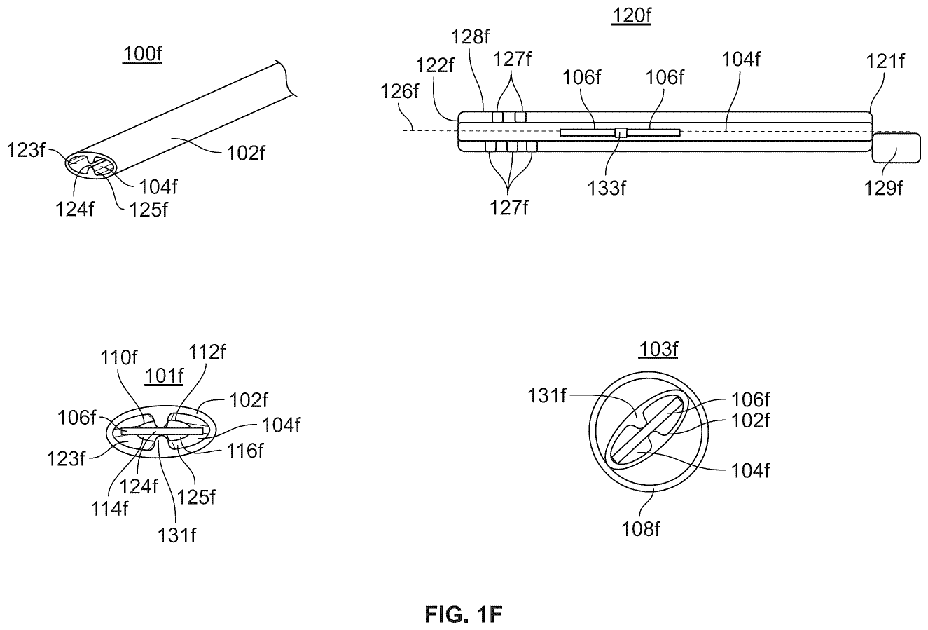

FIG. 1A illustrates an ablation system 100, in accordance with embodiments of the present specification. The ablation system comprises a catheter 10 having at least one first distal attachment or positioning element 11 and an internal heating chamber 18, disposed within a lumen of the catheter 10 and configured to heat a fluid provided to the catheter 10 to change said fluid to a vapor for ablation therapy. In some embodiments, the catheter 10 is made of or covered with an insulated material to prevent the escape of ablative energy from the catheter body. The catheter 10 comprises one or more infusion ports 12 for the infusion of ablative agent, such as steam. In some embodiments, the one or more infusion ports 12 comprises a single infusion port at the distal end of a needle. In some embodiments, the catheter includes a second positioning element 13 proximal to the infusion ports 12. In various embodiments, the first distal attachment or positioning element 11 and second positioning element 13 may be any one of a disc, hood, cap, or inflatable balloon. In embodiments, catheter 10 shaft between first distal attachment or positioning element 11 and second positioning element 13 is flexible. Additionally, in embodiments, a section of a length ranging from 2 mm to 40 mm, proximal to second positioning element 13 (which is the proximal positioning element) is relatively more flexible than the catheter 10 shaft, to allow for positioning elements 11 and 13 to self-center within a hollow and/or tubular organ and be positioned within a tortuous anatomy. In some embodiments, the first distal attachment or positioning element 11 and second positioning element 13 include pores 19 for the escape of air or ablative agent. A fluid, such as saline, is stored in a reservoir, such as a saline pump 14, connected to the catheter 10. Delivery of the ablative agent is controlled by a controller 15 and treatment is controlled by a treating physician via the controller 15. The controller 15 includes at least one processor 23 in data communication with the saline pump 14 and a catheter connection port 21 in fluid communication with the saline pump 14. In some embodiments, at least one optional sensor 17 monitors changes in an ablation area to guide flow of ablative agent. In some embodiments, optional sensor 17 comprises at least one of a temperature sensor, a pressure sensor and/or an impedance sensor. In some embodiments, the catheter 10 includes a filter 16 with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal 25 in data communication with the controller 15, a switch 27 on the catheter 10, or a switch 29 on the controller 15, for controlling vapor flow. In some embodiments catheter 10 includes a wall longitudinally along at least a portion of its outer surface. The wall contains a cooling element, such as a cooling fluid, to limit the maximum temperature of the outer surfaces of catheter 10.

In one embodiment, a user interface included with the microprocessor 15 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels.

The ablation device also includes safety mechanisms to prevent users from being burned while manipulating the catheter, including insulation, and optionally, cool air flush, cool water flush, and alarms/tones to indicate start and stop of treatment.

Referring to FIG. 1B, the treatment end 100b of the catheter comprises an outer sheath that, when pulled proximally toward the clinician, unveils the positioning elements 125b, 115b and wire mesh structure 120b connecting the two positioning elements 125b, 115b. When positioned within the outer sheath, the positioning elements 125b, 115b and connecting wire mesh structure 120b are substantially pressed against the catheter body in a linear configuration. When expanded, the two positioning elements 125b, 115b form wire discs or cones and the wire mesh structure 120b is attached to the distal and proximal positioning elements 125b, 115b at various points along their respective outer peripheries. In some embodiments, the positioning elements 125b, 115b and the wire mesh structure 120b are made of nitinol wires, ranging from 0.1 mm to 0.4 mm in diameter. In some embodiments, 0.22 mm diameter super elastic nitinol is used for the wires.

A first wire or first member 105b positioned within the catheter lumen is fixedly attached to a proximal end or collar 110b of the proximal positioning element 115b. Passing through or attached to the first wire or member 105b is a second wire or member 135b that is fixedly attached to a distal assembly 130b of the distal positioning element 125b. The distal assembly 130b is configured to slide relative to the second wire or member 135b as it is moved proximally or distally. The relative movement of the distal assembly 130b over the second wire 135b helps move the distal positioning element 125b relative to the proximal positioning element 115b thereby expanding the wire mesh structure 120b away from the central lumen where ports 145b are located and toward a tissue surface. The ability of the distal positioning element 125b to slide over the second wire 135b allows the mesh to collapse and for the positioning elements to have a lower profile (smaller diameter). The mesh structure does not fold over the top of itself if the distal positioning element can slide along a rail wire. In other embodiments, the distal positioning element is fixed and does not slide and the wire mesh folds over itself, which increases the collapse diameter. The expanded diameter of wire mesh structure 120b is in a range of 5 mm to 50 mm, and preferably within a range of 20-30 mm, and further preferably of approximately 25 mm. As shown in FIG. 1B, the resulting expanded structure forms a substantially cylindrical or elliptical volume around the centrally positioned catheter body, serving to push tissue away from ports 145b and center ports 145b within the treatment volume. In some embodiments, a length of the cylindrical volume, extending between positioning elements 115b and 125b is in a range of 5 mm to 50 mm, and preferably within a range of 20-30 mm, and further preferably of approximately 25 mm. Preferably the circumference of the wire mesh structure measured at any point along its length is equal to 70 to 100% of the circumference of either positioning element. In certain embodiments, the shape of the wire mesh structure is conical, pyramidal or spherical.

FIG. 1B illustrates a deployed configuration of positioning elements 125b, 115b and wire mesh structure 120b. Alternatively, the combination of positioning elements 125b, 115b and wire mesh structure 120b can be referred to as a single positioning element. Further alternatively, positioning elements 125b, 115b can be referred to as end caps or disks, and wire mesh structure 120b can be referred to as the positioning element or as a central cylindrical region between elements 125b and 115b. Structure 120b is constricted relative to positioning elements 125b, 115b. In embodiments, cylindrical mesh formed by structure 120b can comprise four to eight wire struts crossing at interlaced joints to form diamond-shaped openings. Positioning element 115b is the proximal element and, in embodiments, is attached to a proximal collar 110b. Positioning element 125b is the distal element and, in embodiments, is attached to a distal assembly 130b. In embodiments, the catheter ends distally in a rounded atraumatic tip 140b. The proximal collar and the distal hub are each coupled to at least one member positioned centrally through the catheter shaft. A longitudinal axis can be defined as extending parallel to the longitudinal axis of an internal lumen and passing through positioning elements 125b, 115b and wire mesh structure 120b. To achieve a collapsed (initial) configuration, an outer sheath of the catheter is pushed distally, away from the clinician, over first wire or member 105b, so that second wire or member 135b that is fixedly attached to a distal assembly 130b of the distal positioning element 125b is approached by the outer sheath. Proximal end 150b of wire mesh structure 120b is attached to proximal positioning element 115b, while distal end 155b of wire mesh structure 120b is not fixed, such that pushing of the outer sheath over first wire or member 105b and positioning element 115b results in a sliding movement of the distal end 155b of wire mesh structure 120b along a “rail”. Allowing the distal end 155b to slide along the rail enables wire mesh structure 120b to collapse within the outer sheath of the catheter. If distal end 155b was fixed, the wires of wire mesh structure 120b would have to fold over themselves to collapse into first wire or member 105b, which would result in a larger profile and larger compression forces. In embodiments, each of elements 125b, 115b, and 120b are made using wires and are adapted to expand concurrently and adapted to compress concurrently back into the substantially linear configuration. In embodiments, the collapsed profile of wire mesh structure 120b, in its linear configuration, is as small as possible to fit within the internal diameter of the outer sheath that, in one embodiment, has an outer diameter of approximately 10.5 F (or 3.5 mm). A diameter of the outer sheath of the catheter is limited to a length so that the outer sheath fits inside a minimum working channel diameter of an endoscope, which is 3.7 mm (or can be larger than 3.7 mm). In embodiments, the outer diameter ranges from 5 F to 25 F.

Configurations of ports 145b and positioning elements 125b, 115b and, optionally, the wire mesh tissue control mechanism enabled by deployment of wire mesh structure 120b, a substantially uniform ablation is achieved in the treatment area for each ablation session. The treatment region may be defined by a plurality of sequentially positioned annular rings where each annular ring of the plurality of sequentially positioned annular rings comprises tissue. Each annular ring has an axial length, which may range from 0.05 to 2 mm, an average inner circumference and an average outer circumference where the difference between the average inner circumference and the average outer circumference defines an average thickness of the annular ring.

Uniformity of ablation within the treatment area is a function of an extent of effective ablation measured in three dimensions: first, in terms of contiguity of effective ablation across sequentially positioned annular rings defining the treatment region; second, in terms of contiguity over the internal surface area of each of the annular rings, where the internal surface area is defined by the average inner circumference and the axial length; and third, in terms of an amount of the thickness or variance of the thickness that is ablated.

In one embodiment, the positioning elements, ports, and vapor administration protocol, as collectively described above, are configured to achieve:
1. An effective ablation of at least 50%, preferably at least 60%, preferably at least 70%, and more preferably at least 80%, and up to 100% of the internal surface area of a given annular ring to yield an effectively ablated annular ring;
2. A consecutive sequence of effectively ablated annular rings where the internal surface areas of the consecutive sequence of effectively ablated annular rings comprise at least 50%, preferably at least 60%, preferably at least 70%, more preferably at least 80%, and up to 100% of the total surface area of the treatment region; and
3. For any given one of the consecutive sequence of effectively ablated annular rings, the variance in thickness of the effectively ablated region is no more than plus or minus 25% of the average or mean thickness of the effectively ablated region.

FIG. 1C illustrates an exemplary electrode configuration 100c, in accordance with some embodiments of the present specification. In embodiments, the electrode is configured as a two-layer flexible printed circuit board (PCB) and is configured to be positioned within an internal lumen of an ablation catheter, parallel to a longitudinal axis of the internal lumen. The electrode is configured to receive an electrical current and generate heat to convert a fluid flowing over the electrode into a vapor as an ablative agent. FIG. 1C shows a first top view 110c, a second side view 112c, and a third bottom view 114c of electrode configuration 100c. Electrode configuration 100c is designed with a thin, flexible printed circuit board (flex circuit) 102c using standard PCB fabrication processes and materials. In one embodiment, the base material used for PCB 102c is a sheet polyimide (Kapton). In one embodiment, a thickness of PCB 102c is approximately 0.0254 mm (0.001 inches). In various embodiments, the thickness of PCT 102c ranges from 0.0127 mm to 0.254 mm (0.0005″ to 0.010″). Conductive traces 104c are printed on the top surface 110c and the bottom surface 114c of PCB 102c, resulting in a two-layer PCB. In an embodiment, a total length of PCB 104c is approximately 101.6 mm (4 inches), where the electrode portion comprising traces 104c that extend for approximately 68.58 mm (2.7 inches), can range from 12.7 mm to 914.4 mm (0.5 inches to 36.0 inches) or the full length of the catheter, on the top and bottom surfaces of a distal side of PCB 102c. Traces 104c are shown as the uninsulated traces (exposed surfaces on each side of the flex circuit of PCB 102c). Each conductive trace 104c is fabricated using gold-plated copper. In embodiments, the conductive traces are formed using platinum-iridium or gold. Each conductive trace 104c comprises a first set of anode (+) traces and a second set of cathode (−) traces. The first set of anode traces is separated from another set of anode traces by one of the second set of cathode traces, such that the anode traces and the cathode traces are positioned alternately in parallel or any other formation. Additionally, each conductive trace of alternating polarity pairs can be positioned through the elements 125b and 115b, extending centrally through structure 120b, to form a distributed resistive-heating zone.

In some embodiments, there are four conductive traces 104c traced as exposed electrode surface on each side of PCB 102c, providing eight conductors in total. Two conductive traces 104c on top surface and two on bottom surface, are electrically connected. A fourth view 116c illustrates a blown-out view of electrical connections extending from a proximal side of two conductive traces 104c on the top surface (view 110c), and fifth view 118c illustrates a blown-out view of electrical connections extending from a proximal side of two conductive traces 104c on the bottom surface (view 110c). A sixth view 120c illustrates a blown-out view of PCB 102c when viewed from the side (view 112c) at the point of extending electrical connections from the two conductive traces 104c at the top and bottom surfaces of PCB 102c. In embodiments, the conductive traces on the top and the bottom surfaces have a thickness ranging from 28.35 to 85.05 grams (1 to 3 ounces). Four electrode traces (two electrically connected conductive traces on the top surface and similarly two electrically connected conductive traces on the bottom surface) act as the cathode (+), while the remaining four traces (remaining two electrically connected conductive traces on the top surface and similarly remaining two electrically connected conductive traces on the bottom surface) act as the anode (−). The exposed conductive traces 104c alternate on the top and the bottom surfaces, creating two bipolar pairs on the top and two on the bottom of PCB 102c.

A seventh view 122c illustrates a blown-out view of a distal end of top surface (view 110c) of PCB 102c. View 122c shows that each conductive trace 104c has 0.203 mm (0.008 inches) thickness, and can range from 0.051 mm to 0.762 mm (0.002 inches to 0.030 inches). Each conductive trace 104v is designed parallel to the other, with a gap of approximately 0.203 mm (0.008 inches), which and can range from 0.051 mm to 0.762 mm (0.002 inches to 0.030 inches), between adjacent traces 104v. One of the traces 104v parallel to a first edge of a top/bottom surface of PCB 102v is at a distance of approximately 0.127 mm (0.005 inches) and can range from 0.051 mm to 0.51 mm (0.002 inches to 0.020 inches). From the first edge, while the trace parallel to the opposite edge of the top/bottom surface of PCB 102v is at a distance of approximately 0.330 mm (0.013 inches), which can range from 0.102 mm to 0.762 mm (0.004 inches to 0.030 inches) from the opposite edge. PCB 102v has rounded corners with a radius of approximately 0.254 mm (0.01 inches), and each conductive trace 104v also has rounded distal edges.

FIG. 1D illustrates an electrode configuration circuit 100d, in accordance with some embodiments of the present specification. A first end 102d of circuit 100d provides pathways for conductive traces 104d to electrically connect with a generator (not shown) comprising a power source, where at least one connection is for the cathode and the other is for the anode electrodes. The figure also illustrates solder pads 106d—one on a top surface and one on a bottom surface—where the electrical connections from the conductive traces 104d are soldered onto circuit 100d. One wire (+) is soldered to the top and one wire (−) is soldered to the bottom. The wires from solder pads 106d run through a cable and are electrically connected to the generator. Multi-filar Litz wires (composed of seven insulated smaller wires) are used to reduce impedance and ensure efficient transmission of high-frequency electrical currents. Litz wires reduce the resistance/impedance of the conductive wire for high frequency applications. The figure also shows a portion 108d comprising the exposed conductive traces 104d on circuit 100d. In different embodiments, the two poles of the bi-polar electrode are not side-by-side, as shown. In alternate embodiments, the fluid is configured to flow between the (+) and (−) poles of the electrode. In yet other embodiments, the poles are positioned 180 degree across each other. In some embodiments, the fluid is configured to flow between the two poles of the bipolar electrode, like a sandwich. Furthermore, multiple electrodes, if incorporated, are electrically isolated from each other by one or more dielectric ribs. The dielectric ribs are molded into the opposed elements 125b, 115b, and/or the central region therebetween.

In embodiments, the electrode configuration of FIG. 1C is inserted into a PTFE (Teflon) extrusion that is shaped so there are four fluid paths (tunnels) within the extrusion. PTFE is an exemplary hydrophobic polymer type that is used for the tubing. In other embodiments, different materials may be used that preferably provide a low coefficient of friction and a broad working temperature range. PTFE is known to be the preferred material for use in products such as catheters for delivery channels for medical devices. In various embodiments, alternate extrusion materials, hydrophilic and hydrophobic coatings, and surface treatments, may be used along the microfluidic pathway to optimize flow and surface contact properties of the fluidic system. FIG. 1E illustrates an exemplary drawing of a tubing 102e used to encase the electrode configuration in embodiments of the present specification. FIG. 1E shows a first side view 100e and a second front cross-section view 101e of an elongated tubing 102e. Tubing 102e has an exemplary length of approximately 1,524 mm±25.4 mm (60±1 inches). An external surface 104e of tubing 102e is oval in shape, with a short diameter of approximately 0.940 mm (0.037 inches), and a long diameter of approximately 2.184 mm (0.086 inches).

The PTFE material forming tubing 102e has a wall of thickness of approximately 0.127 mm (0.005 inches), except towards the center of the shorter diameter of tubing 102e, where the wall thickens and extends internally towards the opposite side, resulting in the formation of two tunnels 106e and 108e, defined by an internal surface 110e of a lumen formed by tubing 102e. Each tunnel 106e,108e has two bi-polar pairs of electrodes running within them—one pair on a top surface of the electrode configuration and the other pair on the bottom surface of the electrode configuration, where the electrode configuration along with the PCB comprising the configuration is placed at the center of lumen of tubing 102e. Once the electrode configuration is positioned within tubing 102e, each tunnel 106e and 108e is further divided into two additional sub-tunnels, where each sub-tunnel comprises a pair of bi-polar electrodes. The long diameter of internal lumen of tubing 102e is of a length that approximates 1.93 mm (0.076 inches). The internal lumen bends around a smooth curve of approximately 0.132 mm (0.0052 inches) radius at the top and bottom centers to form smooth depressions of radii of approximately 0.139 mm (0.0055 inches), that provide the internal lumen a shape of a batwing and result in the two tunnels 106e and 108e. The opposing depressions at the center of the internal lumen of tubing 102e, while extending towards each other, do not touch each other, so that a gap of approximately 0.127 mm (0.005 inches) remains between them, which allows positioning of the electrode configuration. The top surface of the electrode configuration faces the depression on the top side of internal lumen of tubing 102e, while the bottom surface of the electrode configuration faces the opposite side.

FIG. 1F shows different views of a tubing 102f with the batwing-shaped internal lumen 104f (described with respect to FIG. 1E) comprising a plurality of electrodes 106f, in accordance with some embodiments of the present specification. A first view 100f shows a three-dimensional perspective view of tubing 102f. A second view 101f shows a front cross-sectional view of tubing 102f with electrode configuration 106f positioned therein. A third view 103f shows a cross-section view of tubing 102f with the plurality of electrodes 106f positioned therein, and placed within an external catheter body or sheath 108f. A fourth view 144f shows a catheter assembly 120f having a tubular body 128f with a proximal end 121f, a distal end 122f, and an internal lumen 104f. In some embodiments, the plurality of electrodes 106f divides internal lumen 104f of tubing 102f into a plurality of channels, for example, four tunnels—110f, 112f, 114f and 116f. During operation, ablation fluid (water or saline) flows through the four tunnels 110f, 112f, 114f and 116f over the exposed electrode surfaces on the top and bottom surfaces of the plurality of electrodes 106f. The fluid itself completes the electrical circuit between each bi-polar pair of electrodes within each tunnel—110f, 112f, 114f and 116f.

PTFE is flexible as a material for tubing 102f. The flexibility of tubing 102f allows for a flexible plurality of electrodes 106f when bent in both the “up” and “down” directions. The “batwing” shaped internal lumen 104f of tubing 102f is designed to not collapse upon bending. The support material on each side of electrode configuration 106f holds tunnels 110f, 112f, 114f and 116f, open, even when bent. The consistent tunnel or channel size in different bend angles allows for a consistent flow over the conductive traces on the plurality of electrodes 106f surfaces. The PTFE used to manufacture tubing 102f has hydrophobic properties that repel fluid and does not easily “wet” by the fluid, since it has a high contact angle in a droplet. The hydrophobic nature of the tubing 102f forces the ablation fluid to preferably wet the surfaces of the plurality of electrodes 106f. Contact of the ablation fluid with the surfaces of the electrodes on the plurality of electrodes 106f enables optimal formation of steam when the electrodes are powered and activated. The fluid channels formed within tunnels 110f, 112f, 114f and 116f, which create the shape of the batwing, enables only a thin layer of ablation fluid to flow over the plurality of electrodes 106f surfaces. The thin layer of the ablation fluid heats faster and more uniformly relative to a thicker layer in a deeper lumen. A thin fluid layer, defined by a small volume of fluid, allows for rapid heating of the fluid and therefore rapid conversion from a liquid to a vapor. The optimum depth of fluid that is configured to flow over the electrode surface of the present specification depends of a variety of system parameters, including electrode geometry (size, shape, length, and distance between poles) voltage, current, and flow rate. In one embodiment, a thickness of a fluid column in contact with each bi-polar electrode pair is in range from 0.1 mm to 1.5 mm. In some embodiments, the thickness of the fluid is in a range of 0.2 mm to 0.3 mm at the thickest location in the fluid paths. In an embodiment, a narrowed lumen through which fluid is configured to flow have a depth or height of less than 0.5 mm. Other depths or thicknesses of the fluid paths are allowed with modifications to other parameters such as voltage, flow rate, electrode surface area, electrode length, and number of bi-polar electrode pairs. The objective of any combination of fluid thickness and other parameters is to optimize the amount of fluid that flows over the electrodes so that maximum amount of and uniform distribution of vapors are obtained. Therefore, other electrode configurations and designs are possible within the scope of the present specification.

The electrode configurations shown in FIGS. 1E and 1F may be used in the devices of the present specification, for example, in the devices shown in FIGS. 1A, 1B, 2A, 2B, 2C, and 3, for the generation of ablative agent. Referring to FIG. 1F, in embodiments, an ablation device of the present specification comprises a catheter assembly 120f having a tubular body 128f with a proximal end 121f, a distal end 122f, and an internal lumen 104f extending therebetween. In embodiments, the internal lumen 104f is defined by a longitudinal axis 126f, wherein at least a portion of the internal lumen 104f has a cross-section defined by opposed arcuate lobes 123f, 125f separated by a central region 124f that is constricted relative to the opposed arcuate lobes 123f, 125f.

In embodiments, a plurality of electrodes 106f extends parallel to the longitudinal axis 126f of the internal lumen 104f and passes through the opposed arcuate lobes 123f, 125f and central region 124f of the lumen 104f, wherein the plurality of electrodes is electrically coupled to a controller 129f and configured to receive radiofrequency energy from the controller to heat a fluid within the lumen 104f to form an ablative vapor. The catheter assembly 120f further includes ports 127f extending along the tubular body 128f that place the internal lumen 104f in communication with an exterior of the catheter assembly 120f, wherein the ports 127f are positioned distal to the plurality of electrodes 106f.

In some embodiments, the opposed arcuate lobes 123f, 125f, in combination with the constricted central region 124f, are configured to concentrate heating of the fluid adjacent the plurality of electrodes 106f while maintaining flow balance between the lobes 123f, 125f to substantially uniformly generate ablative vapor for discharge through one or more of the ports 127f. In various embodiments, the cross-section of the internal lumen 104f is batwing-shaped. In some embodiments, the batwing-shaped lumen is configured to generate counter-rotational vortices of the ablative vapor in the two opposed arcuate lobes to equalize temperature.

In some embodiments, the opposed arcuate lobes 123f, 125f comprise two symmetric lobes joined by a medial web 131f having a thickness less than half of an outer wall thickness of the tubular body 128f.

In some embodiments, the plurality of electrodes 106f comprises thin-film conductive traces deposited on a polymeric layer. In some embodiments, the conductive traces comprise platinum-iridium or gold. In some embodiments, the conductive traces comprise a first set of anode traces and a second set of cathode traces, wherein each of the first set of anode traces is separated from another one of the first set of anode traces by one of the second set of cathode traces. In some embodiments, the plurality of electrodes 106f comprise individual conductive traces of alternating polarity pairs positioned through the lobes 123f, 125f to form a distributed resistive-heating zone.

In embodiments, the constricted central region 124f comprises a micro-channel configured to equalize pressure between the opposed arcuate lobes 123f, 125f. In some embodiments, a surface of the internal lumen comprises a hydrophilic coating. In some embodiments, each electrode of the plurality of electrodes 106f is electrically isolated from each other by one or more dielectric ribs 133f. In some embodiments, the one or more dielectric ribs 133f are molded into the opposed arcuate lobes 123f, 125f and/or the central region 124f.

Referring to FIGS. 1C-1F, saline or fluid passes over the exposed traces, and electricity passes through the conductive fluid (saline conducts electricity due to the salt ions). In embodiments, the electricity has a frequency of 465 kHz. The fluid heats up due to the Ohmic resistive properties of the fluid at this electrical frequency.

The dimensions of the plurality of electrodes 106f and the total conductive trace surface area within the plurality of electrodes 106f, described in the embodiments of the present specification, are optimally designed to convert all of the ablation fluid (passive over the conductive traces) to steam before it exits tubing 102f.

The structure of the batwing tubing 102f and plurality of electrodes 106f may be configured to be positioned within a catheter exterior shaft or sheath 108f.

There is a sensitive balance between the electrode surface area, length, flowrate, flow channel size (depth/volume), voltage, and current. In embodiments, voltage and flow rates are optimized for this electrode assembly design to deliver the best quality of steam (most amount of steam) as possible. These design configurations are variable and can be modified and optimized to suit a requirement. In embodiments, an exposed electrode surface area of the (+) and (−) poles of the bipolar electrode is approximately 69.68 square mm (0.108 square inches), and can range from 6.45 square mm to 1,290 square mm (0.010 sq in to 2.0 sq in). The length of the electrode is 69.34 mm (2.73 inches) in one embodiment, and can range from 25.4 mm to 914.4 mm (1 inch to 36 inches). The flow rate, in an embodiment, is approximately 2.2 ml/min, and can range from 0.5 ml/min to 25 ml/min. While FIGS. 1E and 1F illustrate four channels for fluidic flow, in embodiments, the number of channels could range from one to eight. Each channel has a width ranging from 0.762 mm to 0.889 mm (0.03 inches to 0.035 inches), and height or thickness ranging from 0.254 mm to 0.279 mm (0.010 inches to 0.011 inches). In the illustrated embodiments, the four channels each have a cross sectional area of 0.213 square mm (0.00033 sq inches), which totals to 0.852 square mm (0.00132 sq inches) cross-sectional area for flow across all four channels. In different embodiments, the total cross-sectional area can range from 0.065 square mm to 6.45 square mm (0.0001 to 0.01 sq inches).

Additionally, the voltage is 32V in one embodiment, and can range from 10V to 250V. The current fluctuates during the vaporization process since the impedance changes during steam generation. An average current level is of approximately 3 amps, which range from 0.25 to 12 amps. During periods of large volumes of fluidic flow on the electrode, the impedance is low (in a range of 1-10 ohms) and the current is high (>10 amps). This duration is relatively small as compared to the remaining duration of vapor generation. One the fluid reaches its boiling point, then the impedance increases and the current drops to a level below 1 amp, while more incoming fluid is pushed onto the electrode surface. During steady state vapor generation, the current levels stabilize within a range of 2 to 3 amps.

In various embodiments, ablation therapy is provided to achieve the following therapeutic goals or endpoints for patients with t2DM and laparoscopic sleeve gastrectomy who are on one or more glucose lowering agents with HbA1C levels greater than or equal to 7.5%, and the treatment is considered successful for these patients if any one or more of the following therapeutic goals or endpoints is reached: HbA1C levels are reduced by ≥0.5%; and a dose of the one or more glucose lowering agents is reduced by at least 50%.

### Duodenal Ablation

In some embodiments, treatment is performed in a series of treatments in a proximal to distal direction, starting just distal to the ampulla of Vater and moving distally toward the jejunum. In other embodiments, treatment is performed in a series of treatments in a distal to proximal direction, starting 15-30 cm distal to the ampulla of Vater, near the jejunum, and moving proximally toward the ampulla of Vater. In other embodiments, any combination of treatment sequence, i.e. applying a first ablation near the ampulla of Vater, then treating a very distal 10-30 cm beyond the first treatment, then treating in a distal direction back to the first treatment near the ampulla of Vater. Optionally, in some embodiments, treatment is also applied to the duodenum tissue proximal to the ampulla of Vater. In some embodiments, treatment is applied between the ampulla of Vater and the pylorus. In some embodiments, one of the proximal positioning element or the distal positioning element is positioned to cover the ampulla of Vater, with ablation performed anywhere adjacent and even touching the ampulla. There is no minimum or maximum distance of ablation that must be maintained to or from the ampulla. In embodiments, ablation is performed up to 30 cm to 40 cm distally beyond the ampulla. In various embodiments, each of the treatments areas or ablation zones has a length of approximately 2 cm. For example, in some embodiments, if ablation is applied to a single layer of adjacent treatments, 8 treatments will cover approximately 16 cm in length. In various embodiments, ablation is applied to overlap treatments or ablation zones. Additionally, in embodiments, treatment is applied to come back and treat at a same location already treated. There is no limit to the number of treatments that can be applied. In embodiments, a location is not treated and then immediately treated again. Treatment is first moved to a new location, and then back to a previous location. When treatment is applied to a same location for a second time, it is desirable for the temperature at the same location to have returned to body temperature so that the second treatment is not stacking heat. In some embodiments, following the first ablation at a series of treatment sites, a waiting period of a predefined time is allowed to pass, then a second ablation is performed at the same series of treatment sites. The second ablation is performed at the same dose as the first ablation, or at a higher dose. Finally, the ablated segments are detected and optionally, areas where the ablation was missed or skipped are revisited for a touch-up ablation at the same or at a higher dose than the first or the second ablation.

The ablated tissue is examined for the nature and extent of ablation, preferably after the first and the second ablation. A normal tissue appearance is used to compare the appearance of an ablated tissue to obtain a benchmark for the extent of ablation achieved. A normal tissue without edema has a pale pink or a tan color that is evenly viewed over a circumferential zone of the duodenum. A first type of ablation may be termed as the one causing light bruising or erythema. In the first type of ablation, minimal edema is caused, and the tissue achieves a red or purplish shade of color. A second type of ablation that causes deep bruising results in tissue surface of a purple color with marked edema. In the third type of ablation, marked edema is accompanied with white tissue necrosis (white coagulum). A successful ablation is when the type achieved is between the second and third types of ablation.

When non-ablated regions are identified within the series of treatment zones, those regions are targeted and ablation is selectively performed on them, which is herein termed as touch-up ablations. There are broadly three types of missed lesions where touch-up ablation may be required. In the first type, only unablated patches remain in a treatment zone. Patches are small gaps between ablated regions. Patches are secondary to missed steam ablation holes. Presence of patches only can be considered as an indication of a successful ablation. In some cases, touch-up ablation may not be required. In the second type, large segmental gaps between ablated regions are identified. These gaps may extend from a quarter to a half of a circumferential region within the duodenum. Segmental lesions may result from poor positioning of the ablation device, poor amount of fluid used in the ablation, or from over or under suction. Segmental lesions may or may not require touch-up ablation. The decision to retreat a segmental lesion is based on an extent of ablation achieved in its surroundings, the number of times the region proximate to the segmental lesion has already been ‘touched up’, an extent of the segmental lesion, or ability to position the ablation catheter properly to achieve ablation of the segment. In the third type, termed herein as circumferential lesions, full duodenal sections are missed. All circumferential lesions are re-treated using the touch-up ablation.

All the embodiments of the present specification provide systems, devices, and methods that enable management of diseases such as diabetes, including type 2 diabetes. The treatment systems and methods of the present specification enable diabetes management, which may have been treated previously by the patient with daily insulin and having a first HbA1c level of more than 0.1% and no more than 5%. The treatment systems and methods of the present specification can also enable diabetes management in patients with type 2 diabetes with HbA1c level of less than or equal to 9.0 and more than or equal to 5.5. Such patients on a daily long-acting insulin dose ≤2 U/kg Rx with RFVA are able to stop insulin, or reduce the insulin dose by at least 50%. Embodiments of the present specification provide an alternative to existing treatment methods and devices that result in a therapeutic benefit to selected patient diagnosed with type 2 diabetes that is being treated with daily insulin at a first dosage level and having a first HbA1c level of at least 7.5%, and where the benefit comprises a reduced risk of hypoglycemia wherein the risk of hypoglycemia is reduced to a level of no more than 0.1% occurrence rate of serious hypoglycemic events per year; and the results in reduction of daily insulin to a second dosage level less than the first dosage level and maintains a second HbA1c level that is no greater than the first HbA1c level. Further, metabolic disorder of obesity in such patients with type 2 diabetes and being treated with daily insulin, where an abnormal measure prior to the ablation treatment is an elevated excess body weight, is improved by at least 5%. Additionally, reduced HbA1C levels achieved after the treatment are controlled so that changes in HbA1C are ≤0.5%.

Unlike prior art devices, the present invention can be passed through a conventional endoscope, thereby eliminating the need to manipulate more than one large devices side-by-side. Accordingly, in a preferred use, only the endoscope with the catheter is positioned in the patient's duodenum and no other medical device is required to be used in the duodenum outside the endoscope. Furthermore, because of the small and malleable form factor, the disclosed catheters can be advanced from just adjacent the ampulla of Vater through entire duodenum. The ability to extend through the entire duodenum enables increased ease of coverage, from a minimum of 6 cm to upwards of 15 cm and every increment therein. This effective ablation is achieved by performing 5-6 ablation sessions sequentially, each partially overlapping the previous treatment region.

### Jejunal Ablation

In addition to duodenal ablation, some devices and methods of the present specification are used to treat through ablation, a distance greater than the duodenum, into the jejunum. Unlike prior art devices, devices that reach the length of jejunum can also be passed through a conventional endoscope, thereby eliminating the need to manipulate more than one large devices side-by-side. Catheter devices of the present specification with a diameter less than or equal to 3.5 millimeters (mm) enables the ablation to a longer distance within the small intestine. Accordingly, in a preferred use, only the endoscope with the catheter is positioned in the patient's jejunum and no other medical device is required to be used in the jejunum outside the endoscope. Furthermore, because of the small and malleable form factor, the disclosed catheters can be advanced from just adjacent the ampulla of Vater through entire duodenum and further to a distance of up to 150 cm post ampulla and up to 125 cm post ligament of Treitz. The ability to extend through to a greater length beyond the duodenum and into the jejunum enables increased ease of coverage, from a minimum of 6 cm to upwards of 150 cm and every increment therein. This effective ablation is achieved by performing multiple ablation sessions sequentially, each partially overlapping the previous treatment region.

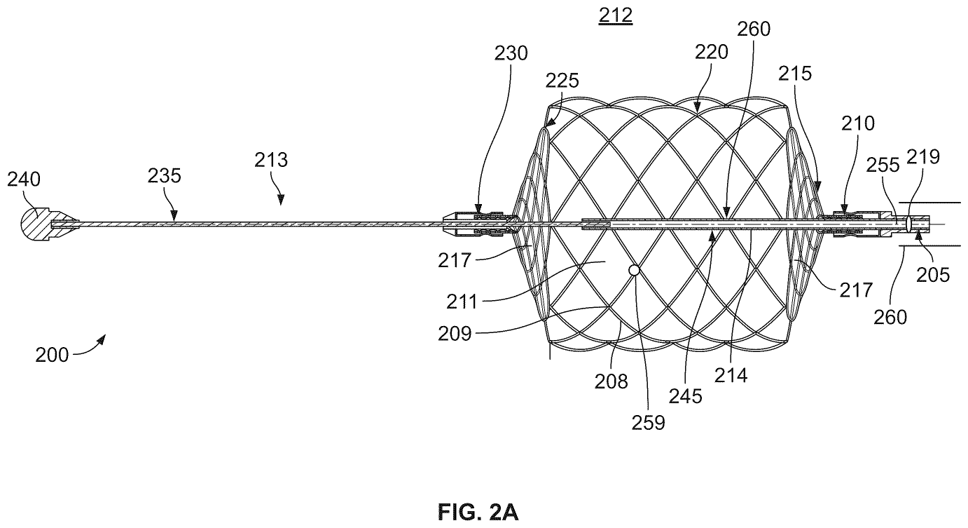

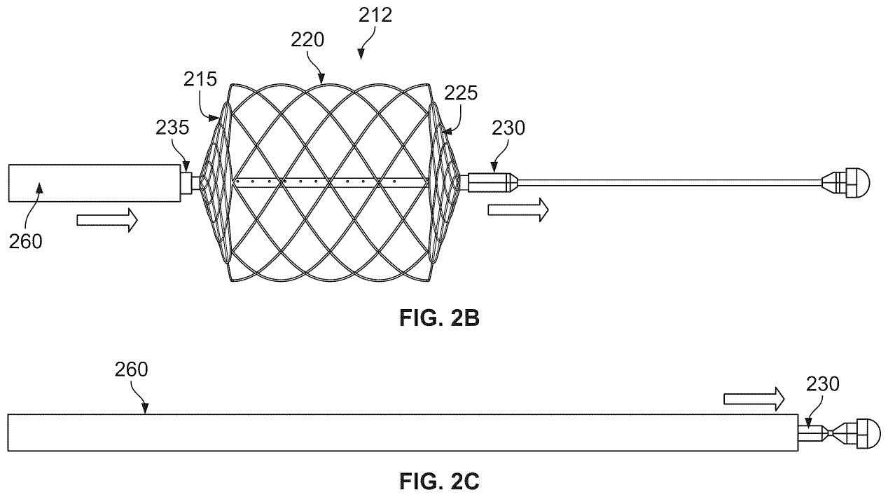

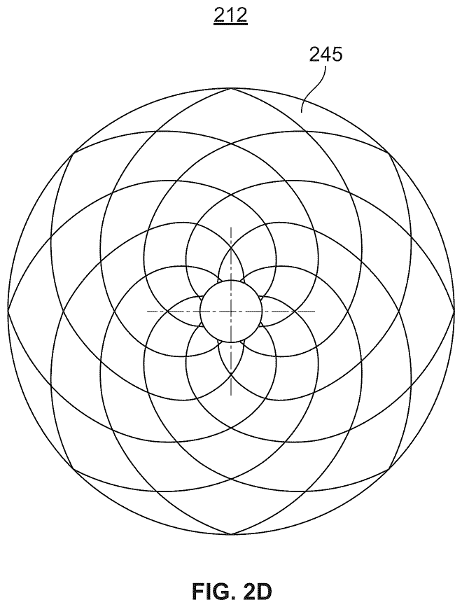

FIG. 2A illustrates the treatment end of a vapor ablation catheter comprising a catheter shaft 214 having a positioning element 212 with a wire mesh structure forming a basket-like shape. A wire or member 235 extends through the catheter shaft 214. Referring to FIG. 2A, in embodiments, a distal treatment section 200 of the catheter 213 comprises an outer sheath 260 that, when pulled proximally toward the clinician (and away from the treatment end 200), unveils a positioning element 212 comprising a wire mesh structure having a proximal end or wire mesh cap 215, a distal end or wire mesh cap 225, and a cylindrical body or wire mesh 220. The catheter 213 has a diameter of less than or equal to 3.5 mm, enabling it to pass through a standard endoscope with a working channel of at least 3.7 mm. When positioned within the outer sheath 260, the positioning element 212 is substantially pressed against the catheter body 214 in a linear configuration. When expanded, the cylindrical body 220 of the positioning element 212 extends away from the catheter body 214. The positioning element 212 approximates a basket or barrel-like shape in an expanded configuration. The structure of positioning element 212 is configured such that it can apply a radial force to the tissue lumen targeted for ablative treatment. In some embodiments, the radial force ranges from 0.025 lb to 1 lb. The positioning element is configured to apply a radial force to the tissue surrounding the lumen to result in a circumferential stretch to the tissue. In some embodiments, the intestinal lumen is larger than the fully expanded diameter of the basket (of approximately 25 mm diameter in one embodiment), and therefore suction is applied to pull the luminal tissue towards the basket, and the basket holds its shape under vacuum. In embodiments, the positioning element 212 is made from nitinol wire(s), ranging from 0.1 mm to 0.4 mm in diameter. In some embodiments, 0.22 mm diameter super elastic nitinol is used for the wires. The positioning element 212 is designed with a wire diameter large enough to provide some structure and resist collapsing of the lumen, even under endoscopic vacuum. In embodiments, each of the distal wire mesh cap 225, proximal wire mesh cap 215, and cylindrical wire mesh 220, is made using a wire mesh and is adapted to expand concurrently and adapted to compress concurrently back into the substantially linear configuration. Each of the distal wire mesh cap 225, proximal wire mesh cap 215, and cylindrical wire mesh 220 can be made using a superelastic alloy, nitinol or stainless steel.

In some embodiments, the wire or member 235 positioned within the catheter shaft 214 is coupled or fixedly attached to a proximal collar 210 which is coupled to the wires of the positioning element 212. For example, wires of the proximal wire mesh cap 215 are coupled to the proximal collar 210 which in turn is coupled to the wire or member 235. In some embodiments, the proximal collar 210 is configured to be slidable along the wire or member 235 to adjust a degree of expansion of the cylindrical wire mesh 220 of the positioning element 212. The wire or member 235 is also coupled or fixedly attached to a distal hub 230 which is coupled to the wires of the positioning element 212. For example, wires of the distal wire mesh cap 225 are coupled to the distal hub 230 which in turn is coupled to the wire or member 235. In some embodiments, the distal hub 230 is configured to be slidable along the wire or member 235 to adjust a degree of expansion of the cylindrical wire mesh 220 of the positioning element 212. In some embodiments, one or both of the proximal collar 210 and the distal assembly 230 are configured to slide relative to the wire or member 235 as it is moved proximally or distally, thereby expanding or collapsing the positioning element 212. In embodiments, distal wire mesh cap 225, proximal wire mesh cap 215, and cylindrical wire mesh 220 are adapted to expand concurrently and adapted to compress concurrently back into the substantially linear configuration. In embodiments, when in the substantially linear configuration, the positioning element 212 is covered by the outer sheath 260 and, when in the expanded configuration, the positioning element 212 is not covered by the outer sheath 260. In some embodiments, the positioning element 212 is configured to automatically expand upon retracting the outer sheath 260 proximally relative to the catheter shaft 214.

The distal wire mesh cap 225, proximal wire mesh cap 215, and cylindrical wire mesh 220 are defined by a plurality of wire members configured to expand outwardly from a substantially linear configuration to a substantially cylindrical mesh to define a treatment zone. In embodiments, the treatment zone has a length in a range of 5 mm to 30 mm. In embodiments, proximal to the positioning element 212 and not shown in FIG. 2A, the catheter includes an electrode or plurality of electrodes within an inner lumen 255, similar to those shown in the configuration in FIGS. 1E and 1F, configured to convert a fluid into vaporized ablative fluid. In embodiments, inner lumen 255 of catheter 213 is shaped similarly to that shown in FIGS. 1E and 1F. The catheter 213 includes a plurality of ports 245 which are positioned within the cylindrical mesh when the positioning element is in the expanded configuration. The ports 245 are configured to discharge vaporized ablative fluid into a volume defined by the substantially cylindrical mesh 220, wherein, upon expansion, the substantially cylindrical mesh is configured to center the catheter shaft 214 within a hollow organ and establish a circumferential contact profile enabling uniform circumferential ablation when the vaporized ablative fluid is released through the ports 245. In some embodiments, the substantially cylindrical mesh 220 comprises four to eight wire struts 208 crossing at interlaced joints 209 to form diamond-shaped openings 211. In some embodiments, each of the wire members of the positioning element 212 comprises an insulated proximal section and an exposed distal section adapted to reduce unwanted heating. In some embodiments, each of the distal wire mesh cap 225 and proximal wire mesh cap 215 comprises inter-wire gaps 217 permitting 1-80% of the vaporized ablative fluid to flow out of the treatment zone.

In some embodiments, the catheter 213 also includes a cooling lumen 205 extending parallel to the catheter shaft 214 and adapted to maintain an external wall of the vapor ablation catheter below 45° C. In some embodiments, at least one of the plurality of wire members comprises a temperature sensor, a pressure sensor, and/or impedance sensor 259. In some embodiments, the distal hub 230 comprises or ends in a rounded, atraumatic tip 240. In some embodiments, at least one of the proximal collar 210, the distal hub 230, and the substantially cylindrical mesh 220 comprises at least one radiopaque marker. In some embodiments, the catheter 213 includes at least one filter element 219 having pores and adapted to generate backpressure.

FIG. 2B illustrates a direction of movement of an outer sheath 260 of the catheter over the wire or member 235, in accordance with some embodiments of the present specification. Outer sheath 260 of the catheter is slid from a proximal end of the catheter towards the direction of the distal end, over the working length of the catheter. In some embodiments, the working length of the catheter is approximately 210 cm, and ranges from 10 cm to 250 cm. In various embodiments, a length of the catheter is dependent on the tissue/indication for ablation.

FIG. 2C illustrates outer sheath 260 positioned over and encompassing the collapsed configuration of positioning element 212. The distal end of outer sheath 260 further pushes distal hub 230 so that assembly 230 also slides until it is blocked by a distal tip of the catheter. At this point, outer sheath 260 encompasses the working length of the catheter. The relative movement of the distal hub 230 over the wire 235 helps move positioning element 212 thereby collapsing wire mesh structure 220 to fit inside outer sheath 260 (when outer sheath 260 is pushed towards the distal direction), or expanding wire mesh structure 220 (when outer sheath 260 is pulled proximally) away from the central lumen where ports 245 are located, and toward a tissue surface. The ability of the positioning element 212 to slide over the wire 235 allows the basket to collapse and have a lower profile (smaller diameter). In some embodiments, the positioning element 212, when collapsed, does not fold over the top of itself if positioning element 212 can slide along a rail wire. In other embodiments, the positioning element 212 is fixed and does not slide and folds over itself when collapsed, which increases the collapse diameter. The sliding movement of the outer sheath 260 in the proximal direction supports the mesh structure of positioning element 212 to self-expand into its basket-like shape when outer sheath 260 is not constraining it. The expanded diameter of positioning element 212 is in a range of 5 mm to 50 mm, and preferably within a range of 20 mm to 30 mm, and further preferably of approximately 25 mm. Additionally, a length of the cylindrical volume, extending between proximate element 215 and distal element 225 is in a range of 5 mm to 50 mm, and preferably within a range of 20 mm to 30 mm, and further preferably of approximately 25 mm, which defines the treatment length in one ablation cycle.

The dimensions of positioning element 212, in embodiments, specifically the diameter of 25 mm and the length of 25 mm, enable optimization of the mechanical characteristics of the positioning element, including stiffness, ease of deployment, and the system's ability to quickly and uniformly fill the treatment chamber with ablative material. It is estimated that a majority of patients have a duodenum diameter of approximately 25 mm to 30 mm (and even up to 34 mm). The jejunum can be slightly smaller but still has a diameter in a range of 22 mm to 30 mm. The dimensions of the present specification are on a lower side and therefore suitable for most patients. Thus, the positioning element mesh 212 always fully expands and is not significantly restricted by the size of the duodenum/jejunum. Further, suction is applied prior to the ablation, to bring the tissue walls down such that they directly contact walls of positioning element 212. Suctioning improves the consistency of steam delivery and uniformity of tissue temperature during ablation, since the diameter of the treatment volume (and therefore distance from the ports) is consistent. In addition, the tissue temperature within the treatment volume would be cooler if a gap were to exist between positioning element 212 and the tissue walls, as a result of steam escaping around the coating on distal elements 215 and 225.

The mesh structure of positioning element 212 is designed so that it does not collapse under vacuum, while it also provides radial force to hold the surrounding tissue. The design of positioning element 212 also provides a controlled or consistent amount of vapor leakage. The wire mesh comprising positioning element 212, with the exception of the surface that is coated, is significantly open so the vapor passes beyond the nitinol wires to contact the tissue with very little surface area blocked by the wires. Additionally, the mesh structure is configured to keep the chamber formed between the proximal wire mesh cap 215 and the distal wire mesh cap 225 at a neutral atmospheric pressure. Procedurally, multiple passes are performed to ensure the treatment of areas that could have been “shadowed” by the wire.

FIG. 2D illustrates a front cross-section view of positioning element 212, in accordance with some embodiments of the present specification. In an embodiment, a diameter of the circle formed by positioning element 212 is approximately 25 mm. In some embodiments, of the total surface area of the cross-sectional surface of positioning element 212 of approximately 490.87 sq mm, approximately 72 sq mm is comprised by open spaces 245 and not covered by the silicone sheet covering (“disk”). The positioning element 212 is configured to not create a perfect seal for vapor within the positioning element 212. The positioning element 212 is configured to constrain and/or slow the passage of vapor to allow heat to be transferred to the target tissue. Open area as a percentage of the total surface area can be represented as approximately 14.67% or almost 15%. In a scenario where luminal tissue is in contact with an outer diameter of positioning element 212 and if suction of the luminal tissue was not blocking the open surface areas, 15% of the end cap area (“disk”) is open allowing steam to escape.

During ablation, the vapor contacts the end caps of the cylinder formed by positioning element 212 as well as the length of the cylinder along its longitudinal axis which in in contact with the target tissue. The surface area of the target treatment is calculated based on 25 mm diameter and 25 mm length of positioning element 212, which is 29.45 cm². As described above with reference to FIG. 2D, the open area is 72 mm² and if the total cylindrical surface area of positioning element 212 including the area the bases 215 and 225 is 2945 mm². In this case, the total percentage of area of the entire cylinder formed by positioning element 212 that is open is (72/2945) 2.44%. Therefore, an amount of vapor that can escape from within positioning element 212 to regions outside the target tissue can range from near zero to 10%.

As shown in FIG. 2A, the resulting expanded structure forms a substantially cylindrical or elliptical volume around the centrally positioned catheter body, serving to push tissue away from ports 245 and center ports 245 within the treatment volume. In some embodiments, a length of the cylindrical volume of positioning element 212, extending between proximal wire mesh cap 215 and distal mesh cap 225 is in a range of 5 mm to 50 mm, and preferably within a range of 20-30 mm, and further preferably of approximately 25 mm. In some embodiments, the shape of wire mesh positioning element 212 may be conical, pyramidal or spherical. In some embodiments, a silicone covering partially coats the conical surface of at least proximal element 215 and can coat both proximal wire mesh cap 215 and distal wire mesh cap 225. In some embodiments, the proximal wire mesh cap 215 forms an angle of 145 degrees with the catheter shaft 214 at the proximal tip of positioning element 212. In some embodiments, the distal wire mesh cap 225 forms an angle of 145 degrees with the catheter shaft 214 at the distal tip of positioning element 212. In some embodiments, the coating covers most of the proximal surface of the proximal wire mesh cap 215, leaving the distal circular edge near the outer periphery of positioning element 212, uncoated. The uncoated portion extends for the length of cylindrical wire mesh 220. In some embodiments, the uncoated portion extends from the circular edge of the cylindrical body 220 that is formed by the junction to the proximal wire mesh cap 215 and distal wire mesh cap 225. The coating adds a layer of safety by protecting the mucosal lining from any thermal injury during the ablation.

FIG. 2A illustrates a deployed configuration of positioning element 212. To achieve a collapsed (initial) configuration, an outer sheath 260 of the catheter is pushed distally, away from the clinician, over the catheter. Wires of proximal wire mesh cap 215 of positioning element 212 are coupled to proximal collar 210, which in turn is coupled to wire or member 235. Wires of distal wire mesh cap 225 of positioning element 212 are coupled to distal hub 230, which is positioned circumferentially around catheter shaft 214 and configured to slide along catheter shaft 214, such that, in some embodiments, pushing of the outer sheath results in a sequential collapse of proximal wire mesh cap 215, cylindrical wire mesh 220, and distal wire mesh cap 225 of positioning element 212 over wire or member 235. Allowing the distal end 225 to slide along the “rail” of wire 235 enables positioning element 212 to collapse within the outer sheath of the catheter. If distal end 225 is fixed, the wires of positioning element 212 would have to fold over themselves to collapse, which would result in a larger profile and larger compression forces. In embodiments, the collapsed profile of positioning element 212 is as small as possible to fit within the internal diameter of the outer sheath that, in one embodiment, has an outer diameter of approximately 10.5 F. In embodiments, the outer diameter ranges from 5 F to 25 F.

Configurations of ports 245 and positioning element 212 and, optionally, the wire mesh tissue control mechanism enabled by deployment of positioning element 212, enable a substantially uniform ablation in the treatment area for each ablation session. The treatment region may be defined by a plurality of sequentially positioned annular rings where each annular ring of the plurality of sequentially positioned annular rings comprises tissue. Each annular ring has an axial length, which may range from 0.05 to 2 mm, an average inner circumference and an average outer circumference where the difference between the average inner circumference and the average outer circumference defines an average thickness of the annular ring.

In embodiments, the wire mesh of the positioning element 212 is made from Nitinol and self-expands to a predetermined shape. In embodiments, the wires of the mesh are coated with silicone, a polymer, PTFE, or ePTFE. The mesh expands to push away intraluminal tissue, so as to ensure that the tissue does not directly touch the vapor emitting that catheter from a location between the two ends—proximal end and distal end—of the mesh. Presence of the mesh avoids uneven heating on the intraluminal cavity. As a result, the compartment created by the mesh looks more like a stent.

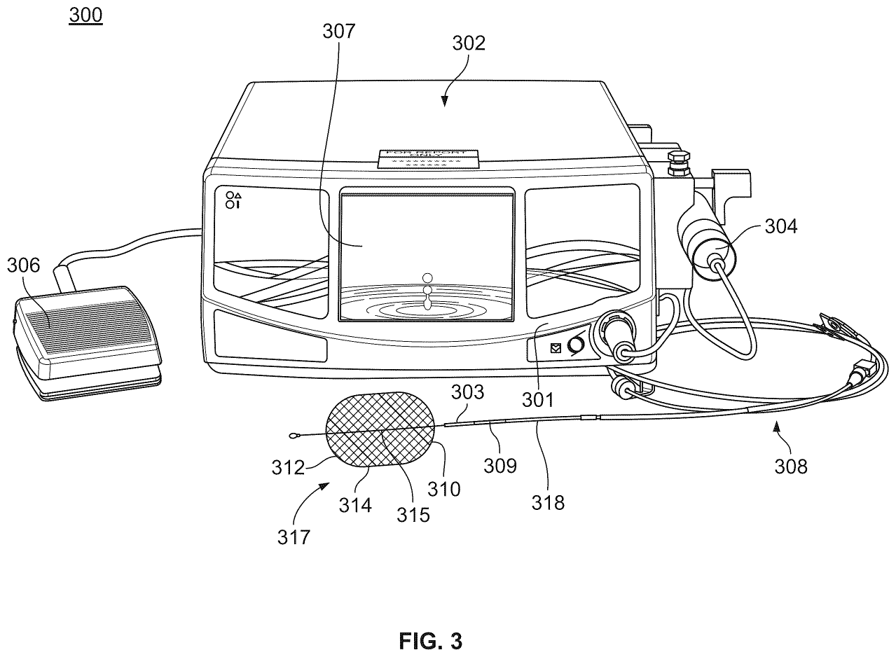

FIG. 3 illustrates an exemplary embodiment of a vapor ablation system 300 in accordance with some embodiments of the present specification. System 300 consists of a controller 302 having a processor and comprising a bipolar RF generator 301 with an integrated syringe pump 304, a foot pedal 306 and a catheter 308. The processor of controller 302 is in electrical communication with catheter 308. Catheter 308 has a proximal end and a distal end, wherein the proximal end is attached to pump 304 and the distal end is configured for insertion in a body cavity. The distal end of circumferential catheter 308 contains a positioning element 317 having a basket comprising a wire mesh structure with a proximal end or wire mesh cap 310, a distal end or wire mesh cap 312, and a cylindrical body or wire mesh 314. It should be noted here that positioning element 317 is a single porous structure that includes ends 310 and 312 in its structure. Ends 310 and 312 are disc shaped or conical as described in context of FIG. 2A. Positioning element 317 is configurable between a compressed pre-deployment configuration and an expanded post-deployment configuration as discussed with reference to FIG. 2A. In some embodiments, a wire mechanism is used to deploy the positioning element 317, as discussed with reference to FIG. 2A. In other embodiments, the wire mesh of the positioning element has shape memory properties and expands automatically when released from a sheath covering the catheter. In some embodiments, both a wire mechanism and shape memory properties are utilized. Ports or spray holes 315 are positioned between ends 310 and 312, which provide an outlet for vapor, which then passes through the spaces in the mesh of positioning element 317 to the target tissue. Once catheter 308 is connected to controller 302 and syringe 304 with saline, bipolar radiofrequency energy (50-60 W) is delivered to an electrode 309 positioned within a lumen 303 in the catheter, for example an electrode or plurality of electrodes within a bat wing shaped lumen as described with reference to FIGS. 1E and 1F. As a fluid, preferably saline, flows over the electrode, heated vapor is generated, for example, at a temperature of 100° C., and delivered to the target tissue through ports 315 and through the mesh of positioning element 317 at the distal end of catheter 308. The wire mesh of the cylindrical body 314 of positioning element 317 pushes away any tissue that could otherwise touch the vapor emitting portion of catheter 308, and avoids uneven ablation. Dosages of the RFVA system are defined as the duration of vapor delivered in seconds (sec). In embodiments, the dosage is predefined or preset by controller 302. An exemplary preset dosage is defined at an energy level of 250 Joules for a period of 3 seconds, after which the heat automatically cuts off. In embodiments, a user controls the delivery of vapor with foot pedal 306, which is in electrical communication with the controller 302. In some embodiments, system 300 includes an interface (GUI) 307 on the controller 302 or an actuator on the catheter 308 for controlling the delivery of vapor. In embodiments, the catheter body 318 is constructed of laser-cut stainless-steel hypotube, providing a combination of durability and controlled flexibility for navigating tortuous anatomy.

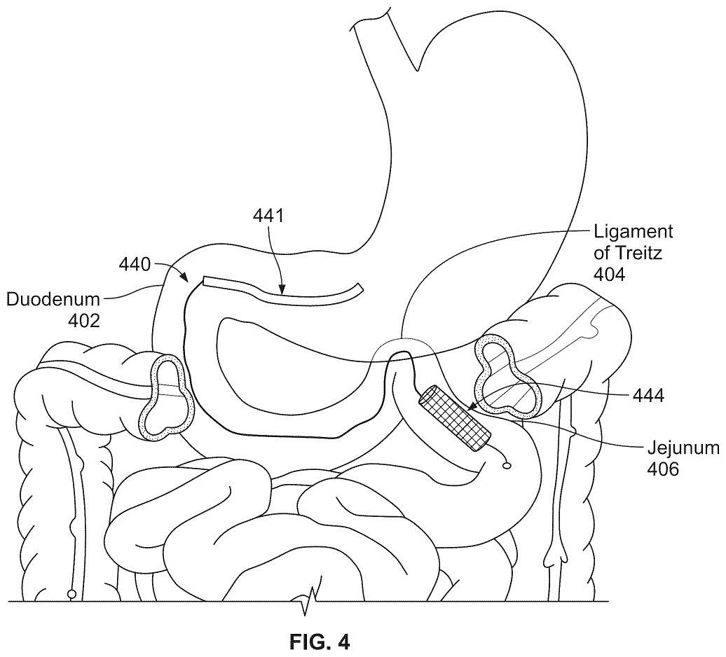

FIG. 4 illustrates positioning of a catheter of the present specification within the jejunum. Referring to FIG. 4, an anatomical view of the small intestine is illustrated. Embodiments of the present specification treat a duodenum 402, and are able to achieve circumferential ablation further from duodenum 402, and even past ligament of Treitz 404, and within a length of jejunum 406.

In some embodiments, the duodenum is treated for a distance of approximately 15 cm past the ampulla. In some embodiments, circumferential ablation of the small intestine is achieved by treating the jejunum 406 starting at a distance of 15 cm and beyond from ampulla. In some cases a distance of up to 150 cm post ampulla is treated. In some cases, distance of more than 2 cm post ligament of Treitz 404 is treated. In some cases up to 125 cm post ligament of Treitz 404 is ablated. In some cases greater than 2 cm of pre ampullary small intestine is ablated by the various embodiments of the present specification. Therefore, the length of ablation achieved by the systems and methods of the present specification is much more than any other known method or device of ablation of small intestine. FIG. 4 further illustrates an ablation catheter 440 (308 of FIG. 3) deployed in jejunum 406 past duodenum 402 and past ligament of Treitz 404 of a patient, in accordance with one embodiment of the present specification. The catheter 440 is deployed through a working channel of an endoscope 441 such that an expandable positioning element 444, shaped like a basket with a mesh structure (212 of FIG. 2 and 317 of FIG. 3) is positioned in jejunum 406.

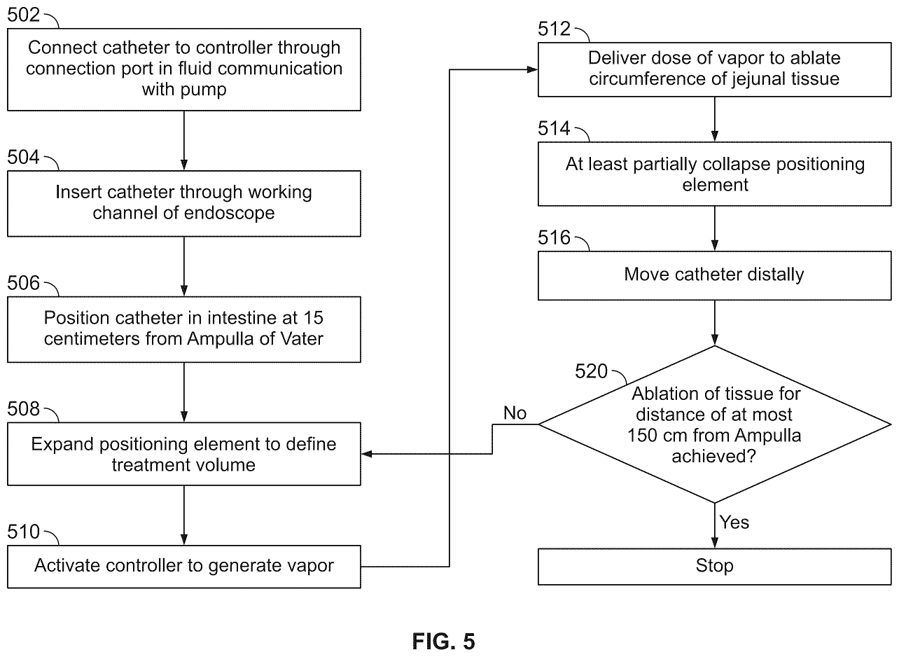

FIG. 5 is a flow chart illustrating an exemplary method of using vapor ablation devices of FIGS. 2-4 for jejunal ablation, in accordance with some embodiments of the present specification. The jejunal ablation is achieved in embodiments of the present specification, by delivering a series of therapeutic doses to a target tissue of the jejunum. When a series of doses are applied to the same target tissue, the effect of the doses aggregate to ablate just enough of a surface area of the mucosa layer. At step 502, a proximal end of a catheter is connected to a catheter connection port to place catheter in fluid communication with a pump. As described previously, a controller having at least one processor is in data communication with at least one pump, which in some embodiments is a syringe pump, and the catheter connection port that is in fluid communication with the pump. The catheter includes a positioning element at its distal end. The positioning element is made from a mesh of Nitinol and is expandable to achieve a cylindrical shape. The enclosed positioning element if formed with a mesh cylinder sealed by a proximal and a distal end. The two ends are also made using a mesh of Nitinol, and are at least partially covered with an insulating material such as, for example, silicone. Each end is disc shaped or conical shaped. One or more ports are positioned in the length of the catheter within the positioning element between its two ends. At step 504, the catheter is inserted through a working channel of an endoscope. In embodiments, the catheter has a diameter of ≤3.5 millimeters (mm), so that it can easily pass through a standard endoscopic channel of up to 3.7 mm. At step 506, the catheter is positioned inside the intestine of a patient, at least 15 cm past the ampulla of Vater or at least 2 cm past the ligament of Treitz. The positioning element is originally in its initial configuration where it is compressed or collapsed within the catheter. At step 508, upon positioning the catheter, the positioning element is deployed to an expanded (second) configuration. In some embodiments, the positioning element forms a treatment zone upon expansion. The length of the treatment zone depends on the length of the cylinder formed by the positioning element. In one embodiment, the treatment zone is approximately 25 mm. A surface area of the proximal end and the distal end of the positioning element comprise a plurality of spaces sufficient to permit a flow of vapor outside of the treatment volume in a range of 1 to 80% of a vapor input flow rate. At step 510, the controller is activated. Activation causes pump to deliver saline into a lumen in the catheter. Additionally, upon activation, the controller causes an electrical current to be delivered to an electrode positioned within the lumen of the catheter. The electrode is located at a place different from the positioning element. Consequently, vapor is generated from the saline at the vapor flow rate. In embodiments, the controller is set to deliver a dose of 250 Joules of energy for a period of three seconds. At step 512, the generated vapor of the preset dose is delivered through the ports within the positioning element and into the treatment zone. The dose of vapor delivered causes circumferential ablation of the jejunal tissue. At step 514, the positioning element is at least partially collapsed. At step 516, the catheter is advanced distally inside the intestine. The distance of advance is such that the distal edge of the previous treatment zone at least partially overlaps with a proximal edge of the adjacent expected treatment zone. In most cases, the distance is substantially similar to the length of the positioning element, which is 25 mm in one embodiment. Steps 508 to 516 are repeated to ablate a distance of up to 150 cm from the ampulla. Each of the plurality of treatment zones at least partially overlaps with a neighboring treatment zone during each repetition, such that they share in a range of 5% to 95% of their jejunal tissue in common. At step 518, if the intestine has been ablated for up to a distance of 150 cm from the ampulla, or up to 125 cm post ligament of Treitz, the process of ablation is stopped. In some embodiments, the circumferential ablation of each of the treatment zones is repeated two or three times.

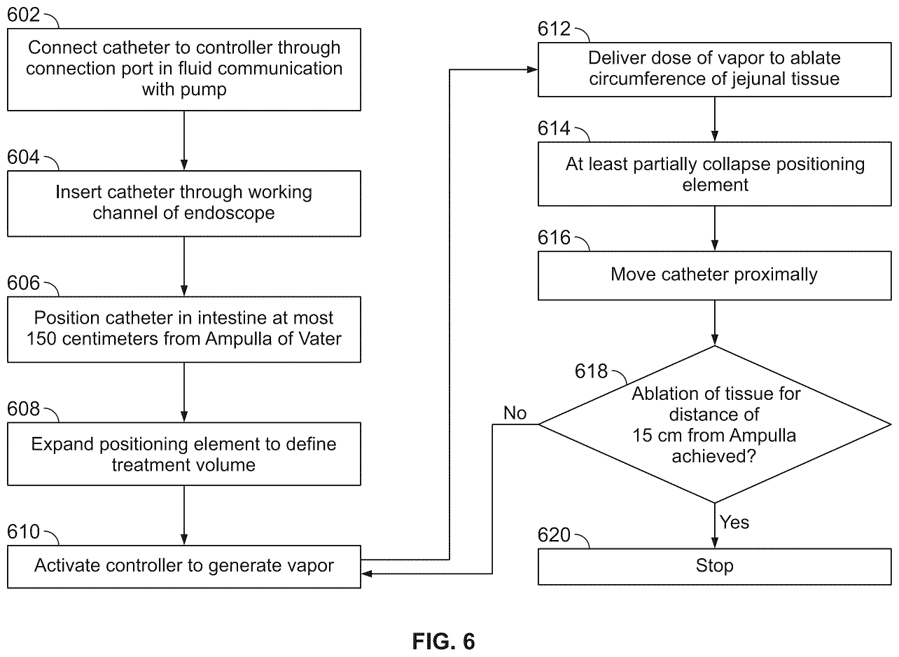

FIG. 6 is a flow chart illustrating an exemplary method of using vapor ablation devices of FIGS. 2-4 for jejunal ablation, in accordance with some embodiments of the present specification. The jejunal ablation is achieved in embodiments of the present specification, by delivering a series of therapeutic doses to a target tissue of the jejunum. When a series of doses are applied to the same target tissue, the effect of the doses aggregate to ablate just enough of a surface area of the mucosa layer. At step 602, a proximal end of a catheter is connected to a catheter connection port to place catheter in fluid communication with a pump. As described previously, a controller having at least one processor is in data communication with at least one pump, which in some embodiments is a syringe pump, and the catheter connection port that is in fluid communication with the pump. The catheter includes a positioning element at its distal end. The positioning element is made from a mesh of Nitinol and is expandable to achieve a cylindrical shape. The enclosed positioning element if formed with a mesh cylinder sealed by a proximal and a distal end. The two ends are also made using a mesh of Nitinol, and are at least partially covered with an insulating material such as, for example, silicone. Each end is disc shaped or conical shaped. One or more ports are positioned in the length of the catheter within the positioning element between its two ends. At step 604, the catheter is inserted through a working channel of an endoscope. In embodiments, the catheter has a diameter of ≤3.5 millimeters (mm), so that it can easily pass through a standard endoscopic channel of up to 3.7 mm. At step 606, the catheter is positioned inside the intestine of a patient, at least 150 cm past the ampulla of Vater or at least 125 cm past the ligament of Treitz. The positioning element is originally in its initial configuration where it is compressed or collapsed within the catheter. At step 608, upon positioning the catheter, the positioning element is deployed to an expanded (second) configuration. In some embodiments, the positioning elements form a treatment zone upon expansion. The length of the treatment zone depends on the length of the cylinder formed by the positioning element. In one embodiment, the treatment zone is approximately 25 mm. A surface area of the proximal end and the distal end of the positioning element comprise a plurality of spaces sufficient to permit a flow of vapor outside of the treatment volume in a range of 1 to 80% of a vapor input flow rate. At step 610, the controller is activated. Activation causes pump to deliver saline into a lumen in the catheter. Additionally, upon activation, the controller causes an electrical current to be delivered to an electrode positioned within the lumen of the catheter. The electrode is located at a place different from the positioning element. Consequently, vapor is generated from the saline at the vapor flow rate. In embodiments, the controller is set to deliver a dose of 250 Joules of energy for a period of three seconds. At step 612, the generated vapor of the preset dose is delivered through the ports within the positioning element and into the treatment zone. The dose of vapor delivered causes circumferential ablation of the jejunal tissue. At step 614, the positioning element is at least partially collapsed. At step 616, the catheter is pulled proximally inside the intestine. The distance of proximal movement is such that the proximal edge of the previous treatment zone at least partially overlaps with a distal edge of the adjacent expected treatment zone. In most cases, the distance is substantially similar to the length of the positioning element, which is 25 mm in one embodiment. Steps 608 to 616 are repeated to ablate a distance of up to 5 cm distal from the ampulla. Each of the plurality of treatment zones at least partially overlaps with a neighboring treatment zone during each repetition, such that they share in a range of 5% to 95% of their jejunal tissue in common. At step 618, if the intestine has been ablated for up to a distance of 5 cm distal from the ampulla, or up to 2 cm post ligament of Treitz, the process of ablation is stopped. In embodiments, another ablation is performed within a treatment zone at a distance of more than 2 cm proximal to the ampulla. In some embodiments, the circumferential ablation of each treatment zone is repeated two or three times.

There are several advantages of ablating the small intestine post ligament of Treitz (which is the jejunum). An Ablation treatment covering a distance of more than 15 cm or into the jejunum improves the acute blood sugar by double the amount relative to that achieved with the ablation of the duodenum alone. A 20% higher improvement was observed in fasting blood glucose levels or HbA1C compared to anything less than these. The longer length of ablation within the small intestine enables better and more durable control over the glycemic index. The higher circumferential ablation achieved by the embodiments of the present specification also result in better and more durable control over the glycemic index. Additionally, overlapping treatments, where the ablation treatment is repeated twice or thrice, results in better and more durable results. During treatments performed with the embodiments of the present specification, on patients with type-2 diabetes with a HbA1C <9.0 and >5.5 n and requiring insulin on a daily long-acting dose of ≤2 U/kg Rx, it was observed that the insulin is stopped, or dose reduced by at least 50%.

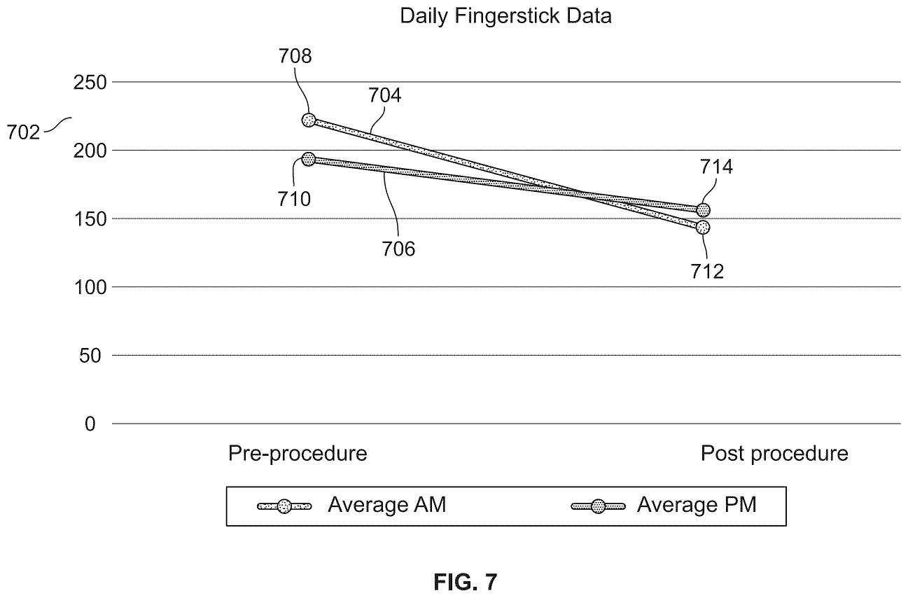

FIG. 7 is a graph 700 illustrating daily fingerstick data of glucose measurement showing the readings for morning and evening, before the treatment and after the treatment, in accordance with the embodiments of the present specification. The data shows an average of the glucose measurements for multiple patients, recorded daily over a period of at least one week. Y-axis 702 of graph 700 shows the measurement made with a fingerstick glucose monitoring device. A first line 704 shows the morning (AM) measurements, whereas a second line 706 shows the evening (PM) measurements. Left-most points 708 and 710 of the first 704 and second 706 lines respectively mark the measurements before the treatment. Right-most points 712 and 714 of the first 704 and second 706 lines respectively mark the measurements after the ablation treatment in accordance with the present specification. It can be observed from graph 700 that average morning measurements show reduction in glucose levels from 222 to 143 (an average change of 79 or 30%), and the average evening measurements show reduction in glucose levels from 193 to 156 (an average change of 37 or 19%). The improvements in the morning glucose measurements using the jejunal ablation methods and system of the present specification are significantly better (an average change of 79 points) when compared to previously known methods of treatment where an ablation treatment covering a length of 12-15cm into the duodenum showed an average improvement of approximately 30 points. Additionally, it was observed that over the same duration as the measurements indicated in the graph, an average pain score for the same set of patients reduced from a level of 2 to a level of 0.

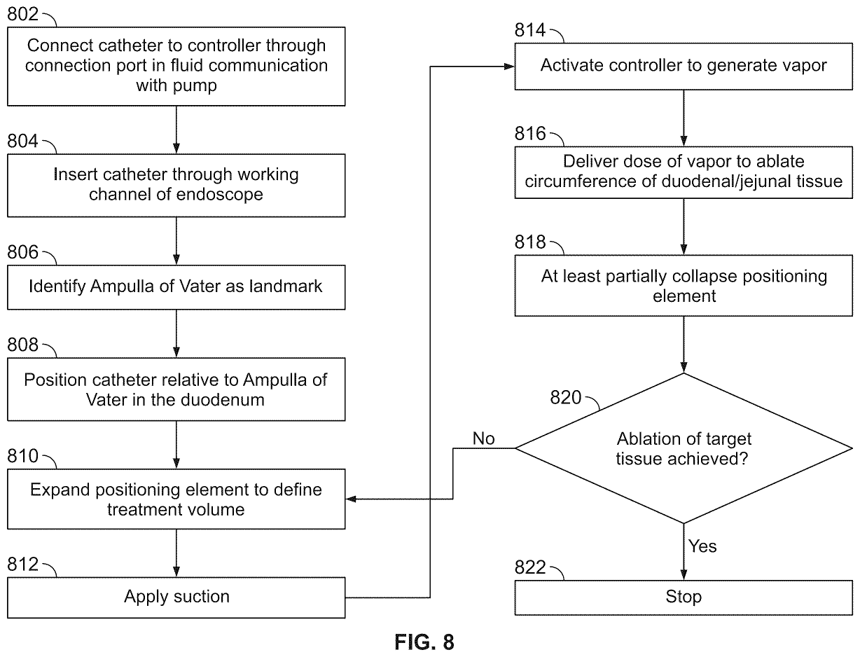

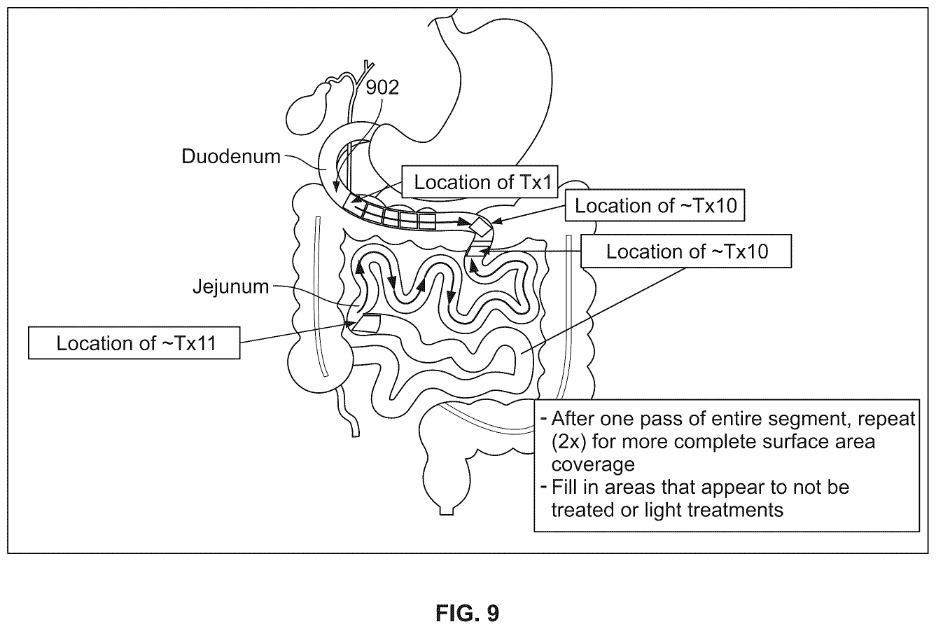

FIG. 8 is a flow chart detailing an exemplary method of using the vapor ablation devices described with respect to FIGS. 2A, 2B, 2C, 3, and 4 for duodenal and jejunal ablation, in accordance with some embodiments of the present specification. The duodenal-jejunal ablation is achieved in embodiments of the present specification, by delivering a series of therapeutic doses to a target tissue of the duodenum and jejunum. When a series of doses are applied to the same target tissue, there is an aggregate effect of the doses that ablate just enough of a surface area of the mucosa layer. At step 802, a proximal end of a catheter is connected to a catheter connection port to place catheter in fluid communication with a pump. As described previously, a controller having at least one processor is in data communication with at least one pump, which in some embodiments is a syringe pump, and the catheter connection port that is in fluid communication with the pump. The catheter includes a positioning element at its distal end. The positioning element is made from a mesh of Nitinol and is expandable to achieve a cylindrical shape. The enclosed positioning element is formed with a mesh cylinder sealed by a proximal and a distal end. The two ends are also made using a mesh of Nitinol, and are at least partially covered with an insulating material such as, for example, silicone. Each end is disc shaped or conical shaped. One or more ports are positioned in the length of the catheter within the positioning element between its two ends. At step 804, the catheter is inserted through a working channel of an endoscope. In embodiments, the catheter has a diameter of ≤3.5 millimeters (mm), so that it can easily pass through a standard endoscopic channel of 3.7 mm or more. At step 806, the Ampulla of Vater is identified and used as a landmark to demarcate the proximal end of a treatment area. FIG. 9 is a pictorial, anatomical view of the digestive system showing the positioning of a clip 902 to mark the proximal end or start of the treatment area. Clip 902 is applied to the contralateral wall to mark the proximal end of the treatment area.

At step 808, a distal end of the catheter is positioned at a distal side of clip 902, at least 15 cm past the ampulla of Vater or at least 2 cm past the ligament of Treitz. The positioning element is originally in its initial configuration where it is compressed or collapsed within the catheter. At step 810, upon positioning the catheter, the positioning element is deployed to an expanded (second) configuration. In some embodiments, the positioning element forms a treatment zone upon expansion. The length of the treatment zone depends on the length of the cylinder formed by the positioning element. In one embodiment, the treatment zone is approximately 25 mm. A surface area of the proximal end and the distal end of the positioning element comprise a plurality of spaces sufficient to permit a flow of vapor outside of the treatment volume in a range of 1 to 80% of a vapor input flow rate. At step 812, the controller is activated to apply suction. Suction is applied to bring the duodenal/jejunal tissue into direct contact with the mesh structure of positioning element, before delivering ablation. Suction is a critical step in the procedure as it brings the tissue down to the diameter of positioning element. Contact of the tissue wall with the structure of the positioning element results in improvement in the consistency of ablation delivery. Additionally, the temperature of the tissue is consistent when the diameter of the duodenal/jejunal (and therefore their distance from the ablation ports) is consistent. Temperature of the tissue wall would be cooler if ablation vapor escaped around the proximal and distal ends due to an oversized lumen or due to a large gap around positioning element.

At step 814, the controller is activated for delivery of saline. The activation causes the pump to deliver saline into a lumen in the catheter. Additionally, upon activation, the controller causes an electrical current to be delivered to an electrode positioned within the lumen of the catheter. The electrode is located at a place different from the positioning element. Consequently, vapor is generated from the saline at the vapor flow rate. While RF ablation method is used in some embodiments, energy sources other than RF may be used to achieve the desired effect. Examples of other energy sources include cryoablation, hot water, microwave, laser, HIFU, or electroporation. In embodiments, the controller is set to deliver a dose if energy ranging from 200 J to 500 J, and in one embodiment an energy of 250 Joules of energy for a period of three seconds. In some embodiments, an energy of 275 J is used. In some embodiments, an output power of 60-65 W is used. If the output power is 60 W and the vapor delivery endpoint is set to 275 J, the vapor delivery time is 4.583 seconds (275 J/60 W=4.58 sec). If the catheter outputs an average of 65 W, the steam delivery time will be 4.23 seconds (275 J/65 W=4.23 sec). In embodiments, the controller is programmed to terminate vapor delivery at 300 J with a 60 W catheter output, when the vapor delivery time will be 5.0 seconds (300 J/60 W). The treatment times of each delivery can differ based on the power output during that delivery. In various embodiments, the termination of vapor delivery is based on total energy delivered and not on time.

At step 816, the generated vapor of the preset dose is delivered through the ports within the positioning element and into the treatment zone. The dose of vapor delivered causes circumferential ablation of the duodenal-jejunal tissue. Referring again to FIG. 9, a first location Tx₁ illustrates the location for a first treatment zone, the treatment progresses sequentially while moving distally through multiple treatment zones. In some embodiments, the first treatment zone Tx₁ is just distal to the ampulla of Vater. Location Tx₁₀ represents a tenth treatment zone, and so on. Multiple treatments can be applied in multiple treatment zones after a minimum 5-second pause to achieve the desired therapeutic effect. In some embodiments, the procedure achieves at least 25 Tx while progressing in a distal direction from the ampulla of Vater. Assuming after each delivery in a treatment zone, the ablation apparatus is advanced approximately 2 cm, then deployed for treatment, approximately 50 cm, or more, of intestine length can be targeted with ablation treatment of the present specification. As discussed in previous sections of the present specification, improved surface ablation is observed with multiple “passes” or repeated treatments on the same tissue. The repeated application approach enables enhancement of mucosal surface coverage without increasing the depth of ablation, provided that sufficient time elapses between consecutive treatments. In some embodiments, each treatment zone is targeted once while progressing in a proximal to distal direction, and then the treatment is delivered in multiple treatment zones progressively while moving in a distal to proximal direction, thus achieving multiple applications through the length of the intestine. In the retrieving direction (distal to proximal), the ablation apparatus is deployed, activated for treatment, retracted by a distance of 2 cm, and then deployed for treatment again. Therefore, one round of ‘down and back’ treatment enables two vapor deliveries within each treatment zone. The rest period between consecutive ablations is significant since immediate repeated ablations at the same site can lead to increased tissue necrosis depth, a phenomenon known as “stacking heat,” where the initial tissue temperature exceeds normal body temperature. In some embodiments, a time delay of approximately 30-40 seconds is adequate for the tissue to return to normal temperature. In some embodiments, waiting for one minute or more is preferable.

The treated areas can be visualized using imaging apparatus to spot and treat any tissue surface(s) that the physician perceives did not get a complete ablation treatment in the first down and back application. Areas can be selected for treatment “as needed”.

At step 818, the positioning element is at least partially collapsed. At step 820, the catheter is advanced distally inside the intestine. The distance of advance is such that the distal edge of the previous treatment zone at least partially overlaps with a proximal edge of the adjacent expected treatment zone. In most cases, the distance is substantially similar to the length of the positioning element, which is 25 mm in one embodiment. Steps 808 to 818 are repeated to ablate a distance of up to 50-70 cm of the post-ampulla small bowel, including the entire length of the duodenum (distal to the Ampulla), through/around the Ligament of Treitz, and 30-55 cm of Jejunum. Each of the plurality of treatment zones at least partially overlaps with a neighboring treatment zone during each repetition, such that they share in a range of 5% to 95% of their duodenal/jejunal tissue in common. At step 822, if the intestine has been ablated for up to a distance of 150 cm from the ampulla, or up to 125 cm post ligament of Treitz, the process of ablation is stopped. In some embodiments, the circumferential ablation of each of the treatment zones is repeated two or three times.

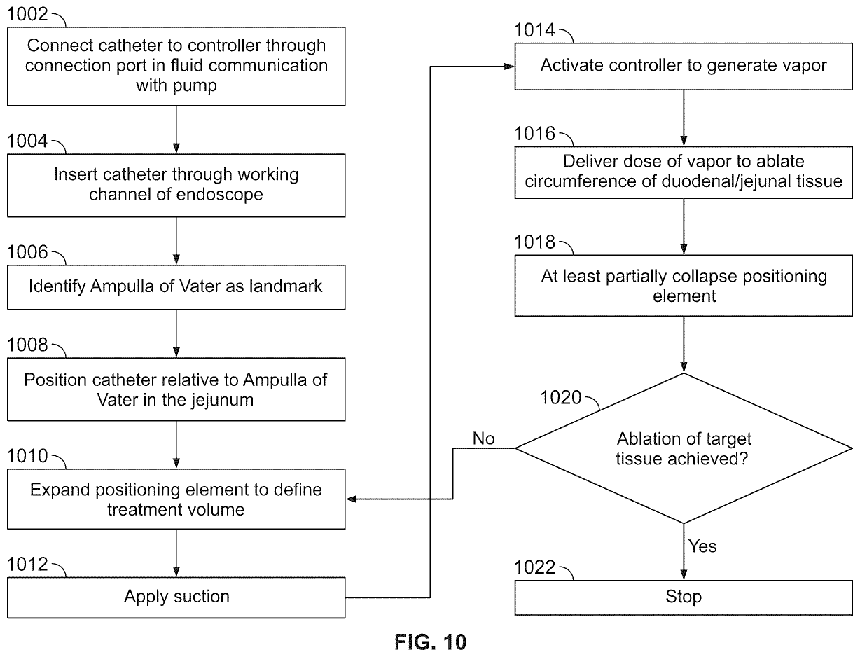

FIG. 10 is a flow chart detailing an exemplary method of using the vapor ablation devices described with respect to FIGS. 2A, 2B, 2C, 3, and 4 for duodenal and jejunal ablation, in accordance with some embodiments of the present specification. The duodenal-jejunal ablation is achieved in embodiments of the present specification, by delivering a series of therapeutic doses to a target tissue of the duodenum and jejunum. When a series of doses are applied to the same target tissue, the aggregate effect of the doses is just enough to ablate a surface area of the mucosa layer. At step 1002, a proximal end of a catheter is connected to a catheter connection port to place catheter in fluid communication with a pump. As described previously, a controller having at least one processor is in data communication with at least one pump, which in some embodiments is a syringe pump, and the catheter connection port that is in fluid communication with the pump. The catheter includes a positioning element at its distal end. The positioning element is made from a mesh of Nitinol and is expandable to achieve a cylindrical shape. The enclosed positioning element is formed with a mesh cylinder sealed by a proximal and a distal end. The two ends are also made using a mesh of Nitinol, and are at least partially covered with an insulating material such as, for example, silicone. Each end is disc shaped or conical shaped. One or more ports are positioned in the length of the catheter within the positioning element between its two ends. At step 1004, the catheter is inserted through a working channel of an endoscope. In embodiments, the catheter has a diameter of ≤3.5 millimeters (mm), so that it can easily pass through a standard endoscopic channel of 3.7 mm or more. At step 1006, the Ampulla of Vater is identified and used as a landmark to demarcate the proximal end of a treatment area. FIG. 9 illustrates an anatomical view of the digestive system showing the positioning of a clip 902 to mark the proximal end or the start of the treatment area. Clip 902 is applied to the contralateral wall to mark the proximal end of the treatment area.

At step 1008, a distal end of the catheter is positioned at a distal side of the clip, at least 30-55 cm after the start of the jejunum. The positioning element is originally in its initial configuration where it is compressed or collapsed within the catheter. At step 1010, upon positioning the catheter, the positioning element is deployed to an expanded (second) configuration. In some embodiments, the positioning element forms a treatment zone upon expansion. The length of the treatment zone depends on the length of the cylinder formed by the positioning element. In one embodiment, the treatment zone is approximately 25 mm. A surface area of the proximal end and the distal end of the positioning element comprise a plurality of spaces sufficient to permit a flow of vapor outside of the treatment volume in a range of 1 to 80% of a vapor input flow rate. At step 1012, the controller is activated to apply suction. Suction is applied to bring the duodenal/jejunal tissue into direct contact with the mesh structure of positioning element, before delivering ablation. Suction is a critical step in the procedure as it brings the tissue down to the diameter of positioning element. Contact of the tissue wall with the structure of the positioning element results in improvement in the consistency of ablation delivery. Additionally, the temperature of the tissue is consistent when the diameter of the duodenal/jejunal (and therefore their distance from the ablation ports) is consistent. Temperature of the tissue wall would be cooler if ablation vapor escaped around the proximal and distal ends due to an oversized lumen or due to a large gap around positioning element.

At step 1014, the controller is activated for delivery of saline. Activation causes pump to deliver saline into a lumen in the catheter. Additionally, upon activation, the controller causes an electrical current to be delivered to an electrode positioned within the lumen of the catheter. The electrode is located at a place different from the positioning element. Consequently, vapor is generated from the saline at the vapor flow rate. While RF ablation method is used in some embodiments, energy sources other than RF may be used to achieve the desired effect. Examples of other energy sources include cryoablation, hot water, microwave, laser, HIFU, or electroporation. In embodiments, the controller is set to deliver a dose if energy ranging from 200 J to 500 J, and in one embodiment an energy of 250 Joules of energy for a period of three seconds. In some embodiments, an energy of 275 J is used. At step 1016, the generated vapor of the preset dose is delivered through the ports within the positioning element and into the treatment zone. The dose of vapor delivered causes circumferential ablation of the duodenal-jejunal tissue. Multiple applications can be applied after a minimum 5-second pause to achieve the desired therapeutic effect. As discussed in previous sections of the present specification, improved surface ablation is observed with multiple “passes” or repeated treatments on the same tissue. The repeated application approach enables enhancement of mucosal surface coverage without increasing the depth of ablation, provided that sufficient time elapses between consecutive treatments. The rest period between consecutive ablations is significant since immediate repeated ablations at the same site can lead to increased tissue necrosis depth, a phenomenon known as “stacking heat,” where the initial tissue temperature exceeds normal body temperature. In some embodiments, a time delay of approximately 30-40 seconds is adequate for the tissue to return to normal temperature. In some embodiments, waiting for one minute or more is preferable.

At step 1018, the positioning element is at least partially collapsed. At step 1020, the catheter is advanced proximally inside the intestine. The distance of advance is such that the proximal edge of the previous treatment zone at least partially overlaps with a distal edge of the adjacent expected treatment zone. In most cases, the distance is substantially similar to the length of the positioning element, which is 25 mm in one embodiment. Steps 1010 to 1018 are repeated to ablate a distance of up to at 50-70 cm of the post-ampulla small bowel, including the entire length of the duodenum (distal to the Ampulla), through/around the Ligament of Treitz, and 30-55 cm of Jejunum. Each of the plurality of treatment zones at least partially overlaps with a neighboring treatment zone during each repetition, such that they share in a range of 5% to 95% of their duodenal/jejunal tissue in common. At step 1022, if the intestine has been ablated for up to a distance of 150 cm from the ampulla, or up to 125 cm post ligament of Treitz, the process of ablation is stopped. In some embodiments, the circumferential ablation of each of the treatment zones is repeated two or three times.

After completing two passes, areas that appear untreated or lightly treated are examined, and these gaps are addressed with additional ablation cycles accordingly. Treated areas are identifiable by their white coagulum appearance and may exhibit sloughing from the submucosa. Visuals of the treated tissue using imaging devices are used to determine optimal treatment. The approach of methods of FIGS. 8 and 10 aim to improve outcomes and durability by replicating the effects of a full duodenal/jejunum bypass without the associated risks or side-effects like infection, pain, bleeding, and digestive issues.

The multi-phase method and system of using vapor ablation for duodenal and jejunal ablation can be used to treat type 2 diabetes mellitus (T2DM), obesity, excess weight, eating disorders, metabolic syndrome, diabetes, dyslipidemia, non-alcoholic steatohepatitis (NASH), non-alcoholic fatty liver disease (NAFLD), metabolic dysfunction-associated steatohepatitis (MASH), or polycystic ovary disease. In various embodiments, ablation therapy provided by the vapor ablation systems of the present specification is delivered to treat a variety of conditions and efficacy of treatment is determined by measuring certain physiological parameters, in a range of time from at least six weeks to two years after treatment. If the therapeutic endpoints are not achieved after a period of at least six weeks, ablation therapy is repeated. Physiological parameters are then measured after at least another six weeks, and ablation therapy may be repeated and evaluated in a similar six-week cycle, until the desired therapeutic endpoint is achieved.

### Duodenal Bulb (D1) Ablation

In some embodiments of the present specification, the target treatment area is the post-ampullary duodenum (D2-D4). There is also, however, potential metabolic regulatory roles within the duodenal bulb (D1), which is targeted for ablation either alone or in combination with either duodenal or duodenal-jejunal ablation. The duodenal bulb may play a pivotal role in nutrient sensing, gastric emptying, and neurohormonal signaling, which are dysregulated in patients with T2DM. Ablative therapy in the region of the duodenal bulb using the embodiments of the present specification has been shown to significantly improve glucose homeostasis. Controlled RF vapor ablation is utilized to selectively target the duodenal bulb while minimizing risks.

D1 differs significantly from the post-ampullary duodenum (D2-D4) in terms of anatomical structure, blood supply, and function, therefore making it a unique target for metabolic intervention. The blood in D1 is primarily supplied by the gastroduodenal artery, whereas D2-D4 receive a dual blood supply from the celiac trunk and superior mesenteric artery. Further, D1 has fewer Brunner's glands compared to D2, making it more susceptible to acid exposure and ulcer formation. Unlike D2-D4, the duodenal bulb plays a pivotal role in regulating gastric emptying and initial nutrient sensing, influencing postprandial glucose excursions. Therefore, ablation of D1 can improve treatment of T2DM by disrupting aberrant duodenal nutrient sensing that contributes to insulin resistance, by modulating gastric emptying to prevent rapid glucose absorption and excessive postprandial hyperglycemia, and by enhancing neurohormonal feedback to suppress hepatic glucose output.

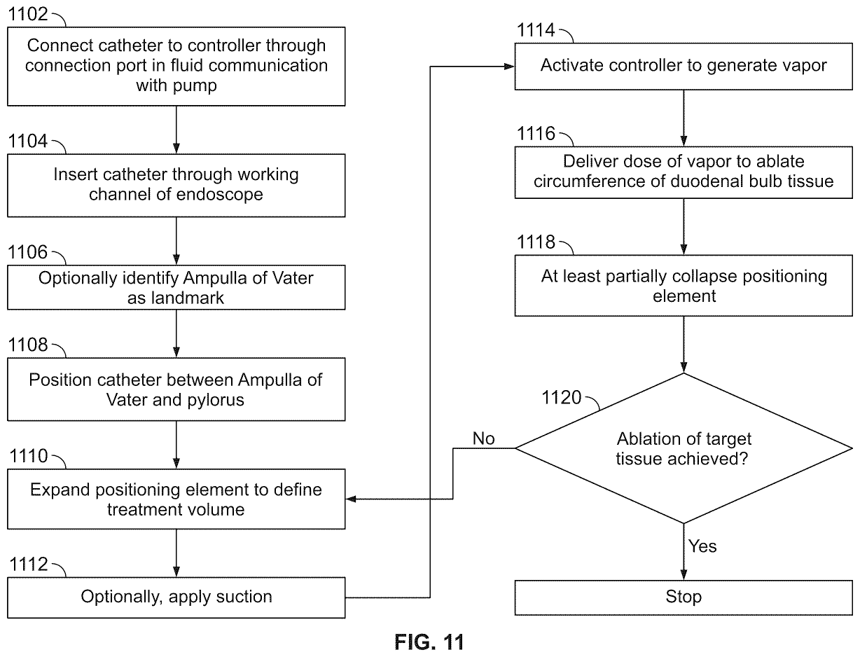

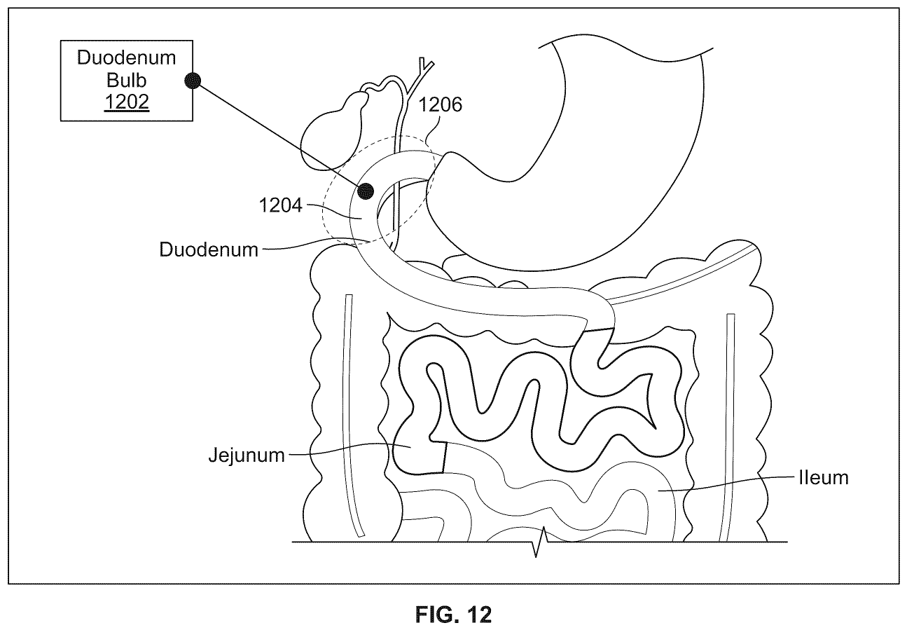

FIG. 11 is a flow chart detailing an exemplary method of using the vapor ablation devices described with respect to FIGS. 2A, 2B, 2C, 3, and 4 for duodenal bulb ablation, in accordance with some embodiments of the present specification. In embodiments, of the present specification, duodenal bulb ablation is achieved by delivering a single or a series of targeted and controlled therapeutic doses within the duodenal bulb. At step 1102, a proximal end of a catheter is connected to a catheter connection port to place catheter in fluid communication with a pump. As described previously, a controller having at least one processor is in data communication with at least one pump, which in some embodiments is a syringe pump, and the catheter connection port that is in fluid communication with the pump. The catheter includes a positioning element at its distal end. The positioning element is made from a mesh of Nitinol and is expandable to achieve a cylindrical shape. The enclosed positioning element is formed with a mesh cylinder sealed by a proximal and a distal end. The two ends are also made using a mesh of Nitinol, and are at least partially covered with an insulating material such as, for example silicone. Each end is disc shaped or conical shaped. One or more ports are positioned in the length of the catheter within the positioning element between its two ends. At step 1104, the catheter is inserted through a working channel of an endoscope. In embodiments, the catheter has a diameter of ≤3.5 millimeters (mm), so that it can easily pass through a standard endoscopic channel of 3.7 mm or more. At step 1106, optionally, the Ampulla of Vater is identified and used as a landmark to demarcate the distal end of a treatment area. FIG. 12 illustrates an anatomical view of a duodenal bulb 1202, which is the segment between an Ampulla of Vater 1204 and a Pylorus 1206.

At step 1108, a distal end of the catheter is positioned at a proximal side of the clip, between the Ampulla of Vater and the pylorus. The positioning element is originally in its initial configuration where it is compressed or collapsed within the catheter. At step 1110, upon positioning the catheter, the positioning element is deployed to an expanded (second) configuration. In some embodiments, the positioning element forms a treatment zone upon expansion. The length of the treatment zone depends on the length of the cylinder formed by the positioning element. In one embodiment, the treatment zone is approximately 25 mm. A surface area of the proximal end and the distal end of the positioning element comprise a plurality of spaces sufficient to permit a flow of vapor outside of the treatment volume in a range of 1 to 80% of a vapor input flow rate. At step 1112, optionally the controller is activated to apply suction. Suction is applied to bring the duodenal/jejunal tissue into direct contact with the mesh structure of positioning element, before delivering ablation.

At step 1114, the controller is activated for delivery of saline. Activation causes pump to deliver saline into a lumen in the catheter. Additionally, upon activation, the controller causes an electrical current to be delivered to an electrode positioned within the lumen of the catheter. The electrode is located at a place different from the positioning element. Consequently, vapor is generated from the saline at the vapor flow rate. While RF ablation method is used in some embodiments, energy sources other than RF may be used to achieve the desired effect. Examples of other energy sources include cryoablation, hot water, microwave, laser, HIFU, or electroporation. Delivery of thermal energy is controlled with accuracy while maintaining safety thresholds. In embodiments, the controller is set to deliver a dose of energy ranging from 200 J to 500 J, and in one embodiment an energy of 250 Joules of energy for a period of three seconds. In some embodiments, an energy of 275 J is used. At step 1116, the generated vapor of the preset dose is delivered through the ports within the positioning element and into the treatment zone. The dose of vapor delivered causes circumferential ablation of the duodenal bulb tissue. Multiple applications can be applied after a minimum 5-second pause to achieve the desired therapeutic effect. As discussed in previous sections of the present specification, improved surface ablation is observed with multiple “passes” or repeated treatments on the same tissue. The repeated application approach enables enhancement of mucosal surface coverage without increasing the depth of ablation, provided that sufficient time elapses between consecutive treatments. The procedure of FIG. 11 comprising delivery of energy are performed under direct endoscopic visualization using a through-the-scope or alongside-the-scope catheter system.

At step 1118, the positioning element is at least partially collapsed. At step 1120, if the duodenal bulb has been ablated, the process of ablation is stopped and the catheter is withdrawn.

Embodiments of the present specification provide a minimally invasive method for treating T2DM through duodenal bulb ablation. These embodiments can be utilized as a standalone therapy for T2DM or in combination with post-ampullary small intestinal ablation or pharmacological interventions (GLP-1 agonists, DPP-4 inhibitors). Ablation of D1 provides enhanced metabolic benefits compared to traditional interventions targeting D2-D4. Unlike endoscopic duodenal resurfacing of D2, which carries a risk of GI bleeding, perforation, and strictures, D1 ablation via RF vapor is minimally invasive and has a lower risk of complications. Controlled RF vapor technology is leveraged to offer superior metabolic regulation compared to existing duodenal interventions while maintaining safety and efficacy. Vapor ablation of the duodenal bulb in accordance with the present specification alters early postprandial glucose excursions by disrupting aberrant nutrient sensing in the duodenal bulb. Additionally, the ablation methods and systems of the present specification are configured to selectively target duodenal mucosa while preserving adjacent structures.

Patients with type 2 diabetes mellitus (t2DM) are sometimes treated with GLP-1 receptor agonists or dual GIP/GLP-1 receptor agonists, which are incretin-based therapies. These medications can help manage blood sugar levels, promote weight loss, and potentially offer cardiovascular benefits. In various embodiments, the ablation therapy is provided to achieve the following therapeutic goals or endpoints for patients with t2DM in combination with post-ampullary small intestinal ablation or pharmacological interventions such as GLP-1 or GIP: reduction in the dose of GLP-1 or GIP by at least 50%; total gain in body weight of ≤5%; and reduced HbA1C levels achieved after the treatment are controlled so that changes in HbA1C are ≤0.5%.

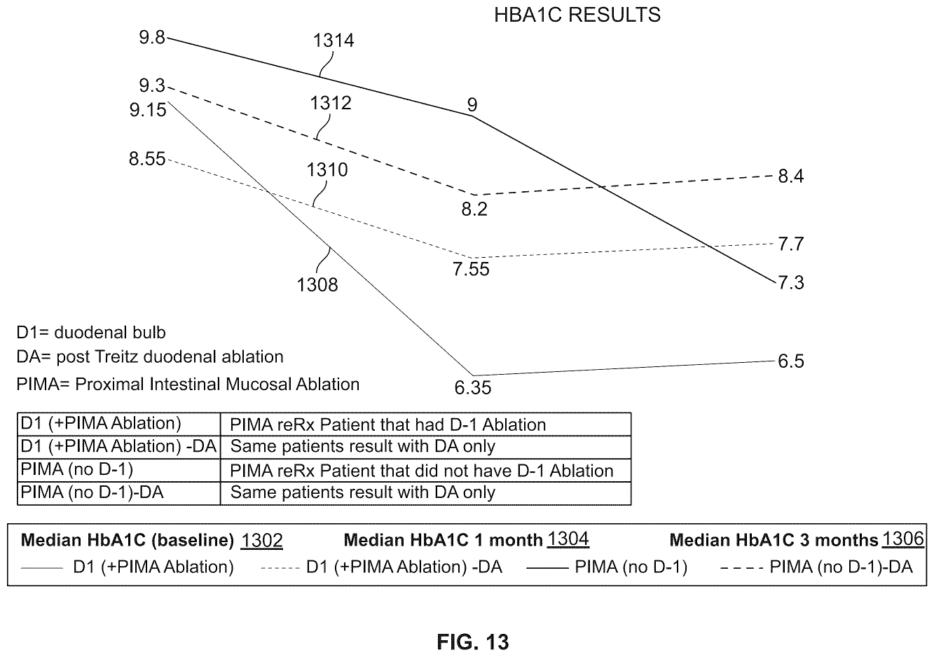

The illustration in FIG. 13 provides a comparative graphical representation of HbA1C levels measured at a baseline 1302, after 1 month 1304 (from baseline 1302), and after 3 months 1306 (from baseline 1302), post-intervention using various combinations of intestinal ablation techniques. The methods compared include: D1 ablation, proximal intestinal mucosal ablation (PIMA), and post Treitz duodenal ablation (DA). The chart aims to demonstrate the efficacy of a combination of these interventions, for improving glycemic control as reflected by reductions in HbA1C levels. The graph is a line chart with three distinct time points: baseline 1302, 1 month 1304, and 3 months 1306. Each line represents the median HbA1C values for a specific intervention groups. The chart uses color-coded and dashed-line distinctions for clarity, where a line 1308 represents D1+PIMA intervention for a first group of patients; a line 1310 represents intervention with only DA for the first group of patients; a line 1312 represents PIMA intervention without D1 for a second group pf patients; and a line 1314 represents intervention with DA only for the second group of patients. The baseline HbA1C levels represented by the median values for all groups range from 9.8 to 8.55. 1 month post intervention, notable reductions are observed across all groups so that the median values for all groups range from 9 to 6.35. 3 months post intervention, while significant improvements in HbA1C are evident, the most pronounced reduction is seen in line 1308 where PIMA intervention was supported with D1 ablation, achieving a median HbA1C of 6.5. This analysis underscores the effectiveness of combining duodenal bulb ablation and proximal intestinal ablation techniques for lowering HbA1C in patients. The chart indicates that the combined use of PIMA and D1 ablation achieves the greatest reduction, suggesting potential synergistic effects of these interventions.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

We claim:

1. A catheter assembly for vapor-based ablation therapy, comprising:
a tubular body having a proximal end, a distal end, and an internal lumen extending therebetween defined by a longitudinal axis, wherein at least a portion of the internal lumen has a cross-section defined by opposed arcuate lobes separated by a central region that is constricted relative to the opposed arcuate lobes;
a plurality of electrodes extending parallel to the longitudinal axis of the internal lumen and passing through the opposed arcuate lobes and central region of the lumen, wherein the plurality of electrodes is electrically coupled to a controller and configured to receive radiofrequency energy from the controller to heat a fluid within the lumen to form an ablative vapor; and
ports extending along the tubular body that place the internal lumen in communication with an exterior of the catheter assembly, wherein the ports are positioned distal to the plurality of electrodes.

2. The catheter assembly of claim 1, wherein the opposed arcuate lobes in combination with the constricted central region are configured to concentrate said heating of the fluid adjacent the plurality of electrodes while maintaining flow balance between the lobes to substantially uniformly generate the ablative vapor for discharge through one or more of said ports.

3. The catheter assembly of claim 1, wherein the cross-section of the internal lumen is batwing-shaped.

4. The catheter assembly of claim 3, wherein the batwing-shaped lumen is configured to generate counter-rotational vortices of the ablative vapor in the two opposed arcuate lobes to equalize temperature.

5. The catheter assembly of claim 1, wherein the opposed arcuate lobes comprise two symmetric lobes joined by a medial web having a thickness less than half of an outer wall thickness of the tubular body.

6. The catheter assembly of claim 1, wherein the plurality of electrodes comprises thin-film conductive traces deposited on a polymeric layer.

7. The catheter assembly of claim 6, wherein the conductive traces comprise platinum-iridium or gold.

8. The catheter assembly of claim 6, wherein the conductive traces comprise a first set of anode traces and a second set of cathode traces and each of the first set of anode traces is separated from another one of the first set of anode traces by one of the second set of cathode traces.

9. The catheter assembly of claim 1, wherein the plurality of electrodes comprise individual conductive traces of alternating polarity pairs positioned through the lobes to form a distributed resistive-heating zone.

10. The catheter assembly of claim 1, wherein the constricted central region comprises a micro-channel configured to equalize pressure between the opposed arcuate lobes.

11. The catheter assembly of claim 1, wherein a surface of the internal lumen comprises a hydrophilic coating.

12. The catheter assembly of claim 1, wherein the plurality of electrodes is electrically isolated from each other by one or more dielectric ribs.

13. The catheter assembly of claim 12, wherein the one or more dielectric ribs are molded into the opposed arcuate lobes and/or the central region.
